(19) United States
(12) Patent Application Publication
(10) Pub. No.: US 2007/0009923 A1
(43) Pub. Date: Jan. 11, 2007

(54) Use of bayesian networks for modeling cell signaling systems

(75) Inventors: Garry P. Nolan, San Francisco, CA (US); Omar D. Perez, San Francisco, CA (US); Karen Sachs, Cambridge, MA (US); Douglas Alan Lauffenburger, Cambridge, MA (US)

Correspondence Address: BOZICEVIC, FIELD & FRANCIS LLP, 1900 UNIVERSITY AVENUE, SUITE 200, EAST PALO ALTO, CA, 94303, US

(73) Assignees: Massachusetts Institute of Technology, Cambridge, MA (US); The Board of Trustees of the Leland Stanford Jonior University, Palo Alto, CA (US)

(21) Appl. No.: 11/338,957

(22) Filed: Jan. 24, 2006

Related U.S. Application Data
(60) Provisional application No. 60/646,757, filed on Jan. 24, 2005.

(51) Int. Cl. C12Q 1/68; G06F 19/00

(52) U.S. Cl. 435/6; 702/20

(57) ABSTRACT

Methods of developing and using models of cellular networks by applying a probabilistic graphical model are provided.

### CROSS-REFERENCE TO RELATED APPLICATIONS

The present application is claims priority to U.S. Provisional Application No. 60/646,757, filed Jan. 24, 2005, which is hereby incorporated by reference in its entirety.

### GOVERNMENTAL SUPPORT OF APPLICATION

This invention was made with governmental support under grant number HV28183 awarded by the National Institutes of Health. The United States government has certain rights in this invention.

### FIELD

The present disclosure discloses experimental and computational methods for constructing cell signaling networks.

### BACKGROUND

Extracellular and/intracellular cues trigger a cascade of information flow, in which signaling molecules become chemically, physically or locationally modified, gain new functional capabilities, and affect subsequent molecules in the cascade, culminating in a phenotypic cellular response. Mapping of signaling pathways typically has involved intuitive inferences arising from aggregating studies of individual pathway components from diverse experimental systems. Although often conceptualized as distinct pathways responding to specific triggers, it is appreciated that discrepant reports of pathway behaviors—especially concerning inter-pathway crosstalk—reflect underlying complexities that cannot be explained by analyses focused on any individual pathway or model system in isolation. To understand cellular responses and their potential dysregulation as implicated in cancer, autoimmunity and other human pathologies, a global, multivariate approach is required (Ideker, T., et al., 2001, Annu. Rev. Genomics Human Gen 2, 343-72).

Bayesian networks, a form of graphical models, have been proffered as a promising framework for modeling complex systems such as cell signaling cascades by representing probabilistic dependence relationships among multiple interacting components (Pearl, J. (1988) Probabilistic reasoning in intelligent systems: networks of plausible inference (Morgan Kaufmann Publishers, San Mateo, Calif.); Friedman, N. (2004) Science 303, 799-805; Friedman, N., Linial, M., Nachman, I. & Pe'er, D. (2000) J Comput Biol 7, 601-20; and Sachs, K., Gifford, D., Jaakkola, T., Sorger, P. & Lauffenburger, D. A. (2002) Sci STKE 2002, PE38). Bayesian network models illustrate the effects of pathway components upon each other in the form of an influence diagram. These models can be derived from experimental data using a statistically founded computational procedure termed network inference. Although the relationships are statistical in nature, they can sometimes be interpreted as causal influence connections when interventional data is used (Pe'er, D., Regev, A., Elidan, G. & Friedman, N. (2001) Bioinformatics 17 Suppl 1, S215-24; Pearl, J. (2000) Causality: Models, Reasoning, and Inference (Cambridge University Press); Hartemink, A. J., Gifford, D. K., Jaakkola, T. S. & Young, R. A. (2001) Pac Symp Biocomput, 422-33; and, Woolf, P. J., Prudhomme, Wendy, Daheron, Laurence, Daley, George & Q. and Lauffenburger, D. A. (2004) Bioinformatics).

### SUMMARY OF CERTAIN EMBODIMENTS

Methods of developing and using models of cellular networks by applying a probabilistic graphical model are provided.

In one aspect, a method of developing a model of cellular networks within a cell category is provided. First cells of said first cell category are contacted with a set of probes that bind to a set of cellular components in each of said first cells, wherein each probe is labeled with a distinguishable label. A plurality of said cellular components in each of said cells is detected to generate a first data set associated with said cellular components in each of the cells. A probabilistic graphical model algorithm is then applied to the data set to identify a first set of arcs between individual cellular components in each of the cells.

The method can further include contacting one or more second cells of the first cell category with an agent. The second cells are then contacted with the set of probes. A plurality of said cellular components in each of the second cells is detected to generate a second data set associated with the cellular components in each of the second cells. A probabilistic graphic model algorithm is applied to the second data set to determine one or more arcs between individual cellular components of the second cell. The first and second sets of arcs are compared to determine the effect of the agent.

In certain embodiments, the decisional arcs identify the agent as therapeutic to the subject. In other embodiments, the decisional arcs identify the agent as toxic to the subject. In still other variations, the first and second cell populations include cells from a subject with a disease state.

The cellular components can be detected using any of a number of techniques. For example, the cellular components can be detected by flow cytometry or confocal microscopy. Any probabilistic graphical model algorithm can be used. For example, the probabilistic graphical model algorithm can be selected from the group consisting of a Bayesian network structure inference algorithm, a factor graph, a Markov random fields model, and a conditional random fields model. In certain embodiments, the probabilistic graphical model algorithm is a Bayesian network structure inference algorithm.

In certain embodiments, cellular components are biological molecules such as proteins (e.g. kinases or phosphatases), substrate molecules, non-protein metabolites (e.g. carbohydrates, phospholipids, fatty acids, steroids, organic acids, and ions).

Arcs can be identified between cellular components that are bound or unbound by the probes. For example, one or more of the arcs can be identified between a cellular components bound by one of the probes and a cellular component not bound by one of the probes. Alternatively, one or more of the arcs can be identified between at least two of the cellular components bound by the probes.

In other embodiments, a method of characterizing a disease state is provided. A first set of arcs for a set of cellular components from measurements of individual cells exhibiting said disease state is provided. A second set of arcs is provided from measurements of individual cells that do not exhibit said disease state. The first and second sets of arcs are compared to determine one or more decisional arcs indicative of said disease state.

In another embodiment, a method of diagnosing a disease state in a subject is provided. A set of decisional arcs indicative of the presence or absence said disease state are provided. A first set of cells are obtained from the subject. A set of probes that bind to a set of cellular components in the first set of cells are provided. Each probe is labeled with a distinguishable label. A plurality of the cellular components in each individual cell of the first set of cells is detected to generate a first data set associated with the cellular components in each of said first cells. A probabilistic graphical model algorithm is then applied to the first data set to identify a set of arcs between individual cellular components in each cell. The set of arcs corresponds to said set of decisional arcs. The disease is diagnosed by comparing the set of arcs to the set of decisional arcs. Prognosis mirrors this approach.

In other embodiments, sub-populations of cells within a given cell population can be identified. A model of cellular networks in each cell in the population of cells are determined. Two or more sub-populations of cells are identified by the presence, absence, or difference in one or more arcs in a first sub-population of said cells as compared to a second sub-population of cells. Individual cells can also be categorized by developing a cellular network, identifying one or more decisional arcs corresponding to each cell category, and categorizing each cell in each of one or more categories.

Methods of refining a model of cellular networks are also provided. Individual cells in a population are categorized into one or more sub-populations of cells. A cellular network is developed in each individual cell. A probabilistic graphical model algorithm is applied to produce a refined model of cellular networks.

Methods of determining the dose of an agent to administer to a subject are also provided. A set of decisional arcs indicative of characteristic of treatment of said disease state pare provided. An agent is then provided to the subject. A set of cells are obtained from the subject, and a set of probes that bind to a set of cellular components in said set of cells are provided to the set of cells. Each probe is labeled with a distinguishable label. A plurality of the cellular components are identified in each individual cell of the set of cells to generate a data set associated with said cellular components in each of said cells. A probabilistic graphical model algorithm is applied to the data set to identify a set of arcs between individual cellular components in each cell. The arcs are compared to the set of decisional arcs to determine the effectiveness of the dose. The dose can be altered based on the effectiveness of the initial dose.

Methods for using computational models for the elucidation of causal connections in cell signaling networks are described herein. The models utilize experimental data obtained from simultaneous multivariate measurements of cellular components present in single cells. For example, a probabilistic modeling algorithm can be applied to determine a graph of causal influences among cellular components in sets of individual cells. Multiple independent perturbation events, such as the addition of agents that can stimulate or inhibit various cellular components comprising a signaling network, can be used to infer the direction of influence between the various signaling components comprising the network. Because each cell is treated as an independent observation, the data provide a statistically large sample that can be used to predict network structure.

The experimental data used to develop models of cell signaling networks generally comprise data obtained from two or more sets of cells, each, comprising cellular components associated with cell signaling networks. Examples of cellular components that can be detected using the methods described herein include, but are not limited to, proteins, scaffold molecules, substrate molecules, and non-protein metabolites, such as carbohydrates, phospholipids, fatty acids, steroids, organic acids, and ions. Multiple observations of the levels of activity of a plurality of cellular components present in individual cells comprising the different sets of cells can be used to generate data sets comprising events associated with the cellular components. Events associated with cellular components, include, but are not limited to, the presence of a given cellular component, changes in the conformation state of one or more proteins (i.e., different structural forms of a protein), changes in the activation state of one or more proteins (i.e., phosphorylation, glycosylation), changes in the concentrations of various cellular components (i.e., cAMP, calcium, mevalonate, glucose, etc.), the redox state of various cellular components (i.e., glutathione, thioredoxin, etc.), cleavage of enzyme substrates (i.e., zymogens, etc.), intracellular quantities of mitogenic indicators (i.e., KI-67, PCNA, histone3-AX, cyclin D, cyclin B, cyclin A, DNA, etc.), and the presence of secondary and/or tertiary RNA structures.

Statistical relationships and dependencies between cellular components can be derived by combining the data obtained from the datasets. For example, Bayesian network analysis can be applied to multivariate flow cytometry data collected using an array of activators and inhibitors to profile the effects of each on the intracellular signaling networks of human primary cells. De novo inferred causal network models can be generated depicting the relationships between the various components comprising the networks. The validity of the models can be evaluated by searching for published reports describing relationships between two or more cellular components in a pathway, or by experimentally verifying the predicted relationships.

In some embodiments, computational models of signaling networks are generated from a first and second set of cells, each, comprising a set of cellular components. Generally, the first set of cells is contacted with a set of probes that bind to a plurality of cellular components present in each of the single cells comprising the first set of cells. A first dataset is generated by detecting the labeled probes bound to the cellular components present in each cell comprising the first set of cells. Agents, capable of altering a plurality of cellular components, are added to the second set of cells. The same set of probes that was used to contact the first set of cells is added to the second set of cells to generate a second dataset. By virtue of the addition of agents that can activate or inhibit the set of cellular components present in the second set of cells, the second dataset differs from the first dataset. The first and second datasets can be analyzed to generate a set of correlations between the different cellular components in the first and second datasets. For example, the analysis can comprise applying a Bayesian network structure inference algorithm to predict causal relationships between a plurality of different cellular components present in the first and second datasets.

Agents capable of altering one or more cellular components include activators, inhibitors and potentiators. The agents used in the methods described herein can be physical (i.e., temperature, pH, salinity, osmolarity, etc.,), chemical (i.e., small molecules such as drugs) or biological (i.e., cytokines, hormones, antibodies, peptides, and protein fragments, either alone or in the context of cells, cells themselves, viruses, nucleic acids, etc.,) in nature.

In other embodiments, different cell types can comprise the first and second sets of cells. For example, in some embodiments, the first and/or second set of cells can comprise cells that are exhibiting a disease state. In other embodiments, the first and/or second set of cells can comprise cells belonging to different tissue types or organs. In yet other embodiments, the first and/or second set of cells can comprise cells that belong to the same tissue type.

Typically, events associated with cellular components are detected using a set of labeled probes. The labeled probes can be selected to bind to a given cellular component. For example, in some embodiments, the labeled probes bind proteins. In other embodiments, the labeled probes bind epitopes associated with a particular conformation or activation state. In other embodiments, the labeled probes can be selected to bind to cellular components that are proteins, proteins, scaffold molecules, substrate molecules, and non-protein metabolites, such as carbohydrates, phospholipids, fatty acids, steroids, organic acids, and ions. Thus, the labelled probes can be selected such that they all bind the same class of cellular component (i.e., proteins), some of them can bind the same class of cellular components, and others can bind a different class of cellular component, or they may all bind different classes of cellular components.

The probes can be labeled with any moiety that, when attached to a probe, renders such a probe detectable using known detection methods, e.g., spectroscopic, photochemical, fluorescent, or electrochemiluminescent methods. For example, in some embodiments, the probes are labeled with a fluorescent moiety capable of generating or providing a detectable fluorescent signal under the specified conditions.

### BRIEF DESCRIPTION OF THE DRAWINGS

FIG. 1A depicts an exemplary embodiment of a signaling network derived from experimental data using Bayesian network analysis.

FIGS. 1B and 1C depict the application of Bayesian networks for hypothetical proteins X, Y, Z, and W.

FIG. 2 depicts consensus network for the illustrated cellular molecules.

FIG. 3A depicts a cell signaling network inferred from flow cytometry data.

FIG. 3B depicts several features of Bayesian networks.

FIGS. 4A-4C depict a model predicting a connection between Erk and Akt (FIG. 4A) and validations for the model (FIGS. 4B and 4C).

FIGS. 5A and 5B depict examples of actual FACS data plotted in prospective co-relationship form.

FIG. 6 depicts correlation connections that pass Bonferroni corrected p value.

FIG. 7 depicts inference results including low confidence arcs.

FIG. 8A depicts a network obtained without the use of activators and inhibitors.

FIG. 8B depicts a network obtained using a population averaged dataset.

FIG. 8C depicts a network obtained using an individual-cell dataset with most of the data randomly excluded to reduce the size of the dataset.

### DETAILED DESCRIPTION

Provided herein are models of cell signaling networks individual cells. The models can be derived from experimental data using one or more probabilistic graphical models. Probabilistic graphical models are graphs showing relationships between nodes (e.g. cellular components). Arcs between cellular components show statistical dependence of the downstream (“second”) cellular component on the upstream (“first”) cellular component. In this context, “upstream” and “downstream” have a directional component; however, arcs generated by the methods of the invention need not have directionality. In certain cases, these statistical dependencies can be interpreted as causal influences from the upstream cellular component upon the downstream cellular component (see, e.g. Pearl, J. (2000) Causality: Models, Reasoning, and Inference (Cambridge University Press).

Several different types of probabilistic graphical models are known in the art. Undirected graphical models, also called Markov Random Fields (MRFs) or Markov networks, have a simple definition of independence. For example, two nodes A and B (or sets of nodes) are conditionally independent given a third set, C, if all paths between the nodes in A and B are separated by a node in C. By contrast, directed graphical models also called Bayesian Networks or Belief Networks (BNs), have a more complicated notion of independence, which takes into account the directionality of the arcs. Discussions of probabilistic graphical models are disclosed, for example, A Brief Introduction to Graphical Models and Bayesian Networks, Kevin Murphy, published 1998, University of British Columbia Website, Department of Computer Science, Kevin Murphy page, and Thesis of Dana Pe'er, School of Computer Science and Engineering, Hebrew University, Israel, each of which is hereby incorporated herein by reference in its entirety. Probabilistic graphical models also include conditional random field models.

Probabilistic graphical models are useful for the inference of signaling networks from biological datasets because they can represent complex stochastic nonlinear relationships among multiple interacting molecules, and their probabilistic nature can accommodate noise inherent to biologically derived data. In addition, probabilistic graphical models can identify direct molecular interactions, as well as indirect influences that proceed via additional, unobserved components, a property crucial for discovering previously unknown effects—including crosstalk between pathways. As described herein, probabilistic graphical models can be used to identify arcs between cellular components in individual cells, thereby eliminating averaging of cellular components.

Bayesian networks are an example of probabilistic graphical models. Bayesian networks have been applied to gene expression data for the study and discovery of genetic regulatory pathways (Friedman, N., Linial, M., Nachman, I. & Pe'er, D. (2000) J Comput Biol 7, 601-20; Pe'er, D., Regev, A., Elidan, G. & Friedman, N. (2001) Bioinformatics 17 Suppl 1, S215-24; Hartemink, A. J., Gifford, D. K., Jaakkola, T. S. & Young, R. A. (2001) Pac Symp Biocomput, 422-33). However, due to the probabilistic nature of the Bayesian modeling approach, effective inference requires many observations of the system. The studies conducted by Friedman et al., supra; Pe'er, et al., supra, and Hartemink et al., supra, utilized lysate-based methods. Bayesian networks derived from lysate-based methods are limited by data sets of insufficient size, and comprise measurements based on averaged samples derived from heterogeneous cell populations, which is a necessary outcome when using lysates from large numbers of cells (Sachs, K., Gifford, D., Jaakkola, T., Sorger, P. & Lauffenburger, D. A. (2002) Sci STKE 2002, PE38; and Woolf, P. J., Prudhomme, Wendy, Daheron, Laurence, Daley, George & Q. and Lauffenburger, D. A. (2004) Bioinformatics).

The methods described herein overcome the limitations associated with lysate-based methods by using detection methods that allow simultaneous observations of multiple cellular components comprising signaling networks in many thousands of individual cells. For example, in some embodiments, intracellular multicolor flow cytometry is used (Herzenberg, L. A., Parks, D., Sahaf, B., Perez, O. & Roederer, M. (2002) Clin Chem 48, 1819-27; and Perez, O. D. & Nolan, G. P. (2002) Nat Biotechnol 20, 155-62.). Intracellular multicolor flow cytometry allows simultaneous observation of multiple cellular components in many thousands of individual cells, and hence, is an especially appropriate source of data for probabilistic graphical models, including Bayesian network modeling of signaling networks. Additionally the use of intracellular multicolor flow cytometry allows for the measurement of biological states in their native contexts. Moreover, unlike mRNA expression profiling, flow cytometry can measure the amount of a protein of interest, and depending upon the technique applied, this can include measures of protein modification states such as phosphorylation (Perez, O. D. & Nolan, G. P. (2002) Nat Biotechnol 20, 155-62; Perez O D, M.D., Jager G C, South S, Murriel C, McBride J, Herzenberg L A, Kinoshita S, Nolan G P. (2003) Nat Immunol 11, 1083-92; Irish J M, H. R., Krutzik P O, Perez O D, Bruserud O, Gjertsen B T, Nolan G P. (2004) Cell 2, 217-28; and U.S. Ser. No. 60/310,141, filed Aug. 2, 2001, 60/304,434, filed Jul. 10, 2001, U.S. Ser. No. 10/193,462, filed Jul. 10, 2002, and U.S. Ser. No. 10/898,734, filed Jul. 21, 2004, all of which are hereby incorporated by reference in their entirety). Since each cell is treated as an independent observation, the flow cytometry data provide a statistically large sample that can enable application of a probabilistic graphical model (e.g. Bayesian network) to accurately predict network structure. Probabilistic graphical models can be used to develop a model of cellular networks within a group or category of cells. The cells of are contacted with a set of probes that bind to a set of cellular components in each of the cells. Each probe is labeled with a distinguishable label. A plurality of cellular components in each individual cell are detected to generate a data set associated with the cellular components in each individual cell. A probabilistic graphical model algorithm is then applied to the data set to identify one or more arcs between individual cellular components in each cell.

Accordingly, provided herein are methods suitable for the multivariate analysis of cellular components present in single cells to generate datasets that can be used to generate cell signaling networks. By “cell signaling network” herein is meant a network comprising two or more cellular components that interact with each other. In certain embodiments, one or more of the cellular components become functionally altered and as a result, gains new functional capabilities that can affect subsequent components in the network. Functional alteration of the cellular components can result from, for example, chemical, physical, or locational modifications.

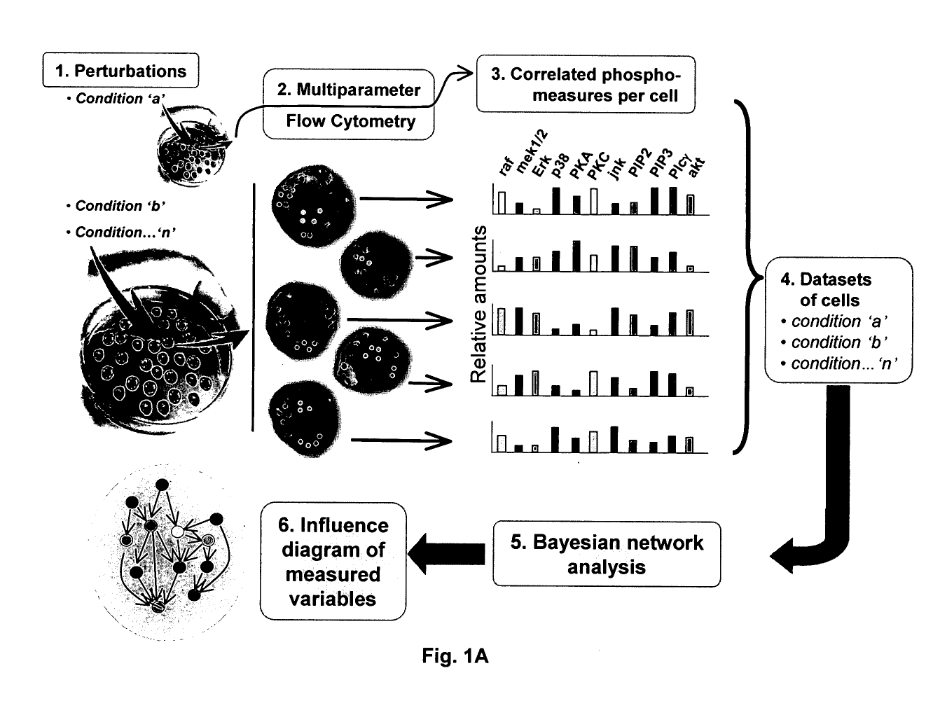

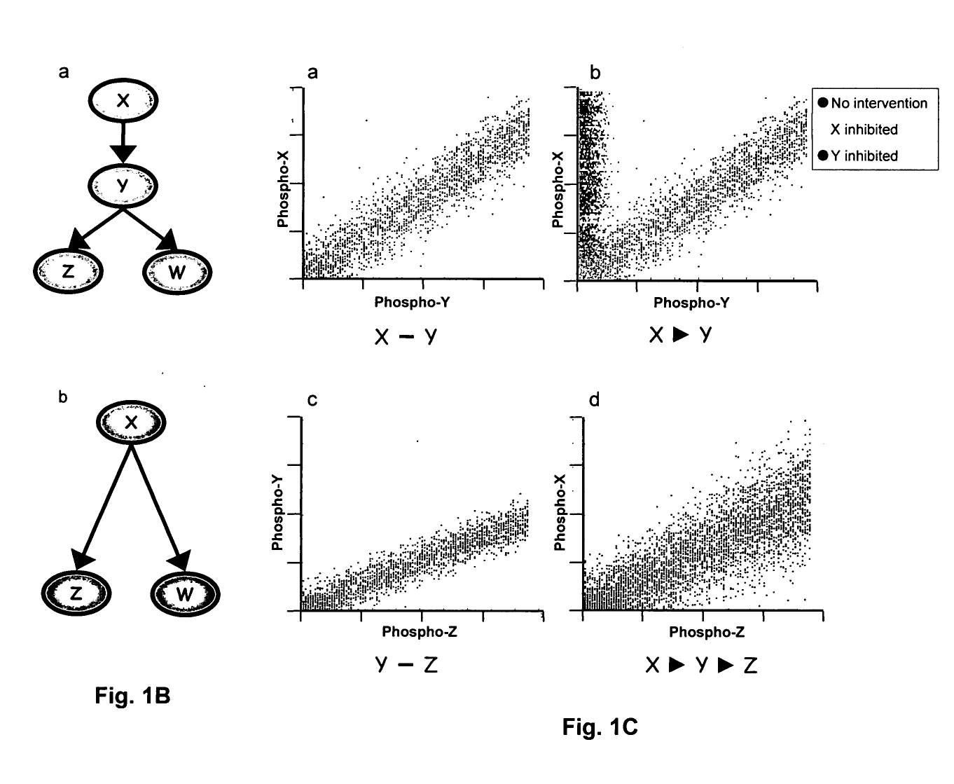

The cellular components can be located in the same pathway, or in different pathways. Thus, in some embodiments, a network can comprise a single pathway, comprising two or more cellular components. The upper panel in FIG. 1B depicts an example of a signaling network represent 4 different hypothetical cellular components located in the same pathway. A directed arc from X to Y indicates that X activates Y, and a directed arc from Y to Z and Y to W indicates that Y activates both Z and W.

The biochemical effects of an agent on cells can be characterized. A model of cellular networks within a group or category of cells can be developed. A second set within the group or category of cells is then provided with an agent. A plurality of cellular components in each cell is detected to generate a second data set. A probabilistic graphical model algorithm is then applied to the second data set to determine a second set of arcs between individual cellular components of the second cells. The first and second sets of arcs are compared to identify a set of one or more decisional arcs indicative of the biochemical effects of the agent.

As used herein, “decisional arcs” refer to arcs used for comparison to other arcs. Decisional arcs can have a value and/or a directionality. The presence, absence, or change in one or more arcs as compared to one or more decisional arcs can determine a change in function of the disease. Decisional arcs can be used, for example, to characterize the biochemical effect of an agent, diagnose a subject with a disease state, or provide a prognosis of a disease state.

An exemplary embodiment of a Bayesian network inference analysis using multidimensional flow cytometry data is depicted in FIG. 1A. In FIG. 1A, an influence diagram (6) depicting correlations between different cellular components can be inferred from individual sets of cells (1). The individual sets of cells can be exposed to different perturbation conditions (1), such as the addition of agents that activate, inhibit, or modulate the cellular components present in the individual sets of cells. The levels of the different cellular components in the individual cells comprising each set (3) can be simultaneously recorded using multiparameter flow cytometry (2). The data obtained from the individual sets of cells can be analyzed using Bayesian network analysis (5) and an influence diagram of the measured components generated (6).

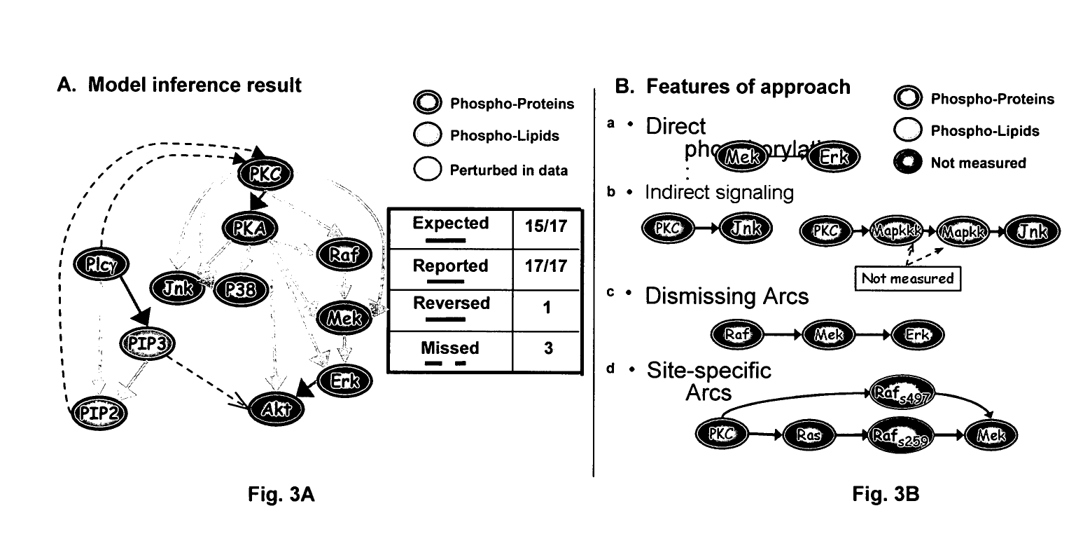

In other embodiments, a network can comprise two or more pathways, each, comprising two or more cellular components, with crosstalk occurring between the cellular components located in the different pathways comprising the network. For example, FIG. 3A depicts an exemplary signaling network comprising three pathways, e.g., Raf to Akt, PKC to P38/JnK, and Plcy to PIP2, with crosstalk occurring between the three different pathways.

The cellular components to be analyzed are typically present in sets of cells comprising individual cells. The number of individual cells in a set can vary, depending in part, on the cellular components to be detected. For example, a set can comprise from 1 to 10, 10², 10³, 10⁴, 10⁵, 10⁶, 10⁷, or 10⁸ cells. The number of sets used in an assay also can vary, depending in part, on the number of agents used agents to derive causal connections between cellular components comprising a signaling network. For example, in some embodiments, two, three, four, five, six, seven, eight, nine, or more sets of cells are used. In other embodiments, from 9 to 100 sets of cells are used. The use of “first”, “second”, etc., in reference to the cell sets disclosed herein, unless specified, is not meant to imply an order or rank.

“Cell categories” or “cell types” are used interchangeably herein to refer to any group of cells defined by a functional or structural characteristic. One advantage of the present invention is that by using data from individual cells, the problems with cell populations is diminished. That is, the techniques used herein will allow identification of cell samples that may accidentally contain more than one cell type (e.g. helper T cells as well as cytotoxic T cells) and distinguish the data accordingly. For example, in some cases the methods of the invention can distinguish between agent effects on different cell types, that is, a different set of decisional arcs will be identified.

Cellular components can comprise any molecule present in a cell that can impact either directly or indirectly a cell signaling network. The term “cellular component” refers to a molecule regardless of molecular weight found within an organism or cell. A cellular component can be from the same class of compounds or from different classes of compounds. Examples of cellular components that can be detected using the methods described herein include, but are not limited to, metabolites, proteins, nucleic acids, carbohydrates, lipids, fatty acids, organic acids, scaffolds, enzyme substrates, cytokines, hormones, organic acids and ions.

“Protein”, “peptide” “polypeptide” and “oligopeptides” are used interchangeably and refer to a polymer of amino acid residues. As used herein, the term “protein” means at least two covalently attached amino acids. The protein may be made up of naturally occurring amino acids and peptide bonds, or, in the case when they are used as agents, synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

By “nucleic acid” or “oligonucleotide” or grammatical equivalents herein means at least two nucleotides covalently linked together. A nucleic acid of the present invention will generally contain phosphodiester bonds, although in some cases, as outlined below, in cases where nucleic acids are used as agents, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

Nucleic acids may be naturally occurring nucleic acids, random nucleic acids, or “biased” random nucleic acids. For example, digests of prokaryotic or eukaryotic genomes may be used as is outlined herein for agent proteins. Where the ultimate expression product is a nucleic acid, at least 10, preferably at least 12, more preferably at least 15, most preferably at least 21 nucleotide positions need to be randomized, with more preferable if the randomization is less than perfect. Similarly, if the ultimate expression product is an protein, at least 5, preferably at least 6, more preferably at least 7 amino acid positions need to be randomized; again, more are preferable if the randomization is less than perfect.

The term “carbohydrate” is meant to include any compound with the general formula (CH₂O)ₙ. Examples of preferred carbohydrates are di-, tri- and oligosaccharides, as well polysaccharides such as glycogen, cellulose, and starches.

The term “lipid” generally refers to substances that are extractable from animal or plant cells by nonpolar solvents. Materials falling within this category include the fatty acids, fats such as the mono-, di- and triacyl glycerides, phosphoglycerides, sphingolipids, waxes, terpenes and steroids. Lipids can also be combined with other classes of molecules to yield lipoproteins, lipoamino acids, lipopolysaccharides, phospholipids, and proteolipids.

“Fatty acids” generally refer to long chain hydrocarbons (e.g., 6 to 28 carbon atoms) terminated at one end by a carboxylic acid group, although the hydrocarbon chain can be as short as a few carbons long (e.g., acetic acid, propionic acid, n-butyric acid). Most typically, the hydrocarbon chain is acyclic, unbranched and contains an even number of carbon atoms, although some naturally occurring fatty acids have an odd number of carbon atoms. Specific examples of fatty acids include caprioic, lauric, myristic, palmitic, stearic and arachidic acids. The hydrocarbon chain can be either saturated or unsaturated.

“Scaffold molecules” generally refer to nucleic acids or proteins that provide a three-dimensional framework to which another molecule can bind.

“Hormones” refer to chemical substances synthesized by endocrine tissue and which act as a messenger to regulate the function of another tissue or organ. Examples of hormones include, but are not limited to, adrenal cortical, adrenocorticotropic hormone (ACTH), antidiuretic hormone, corticosteroid, endocrine human growth hormone and others taught in Lehninger Principles of Biochemistry, 3^{rd} ed, (2000) Worth Publishers, incorporated herein by reference in its entirety.

An “organic acid” refers to any organic molecule having one or more carboxylic acid groups. The organic acid can be of varying length and can be saturated or unsaturated. Examples of organic acids include, but are not limited to, citric acid, pyruvic acid, succinic acid, malic acid, maleic acid, oxalacetic acid, and alpha-ketoglutaric acid. Organic acids can include other function groups in addition to the carboxylic acid group including, for example, hydroxyl, carbonyl and phosphate.

An “ion” refers to an atom or group of atoms that have acquired a net electric charge by gaining or losing one or more electrons. Examples of ions include, but are not limited to, Ca²⁺, Na⁺, Cl⁻, Mg2+, PO₄⁻, and Mn²⁺, etc.

The exact numbers of cellular components and/or pathways that can be identified as belonging to a cell signaling network using the methods described herein will vary, depending in part, on the number of probes used to detect the cellular components, and, in part, on the number of agents used to induce changes in one or more of the cellular components comprising the network. Thus, a cell signaling network can comprise from 2 to 100 cellular components, from 2 to 75 cellular components, from 2 to 50 cellular components, from 2 to 25 cellular components, from 2 to 15 cellular components, from 2 to 10 cellular components, and from 2 to 5 cellular components. As will be appreciated by a person skilled in the art, the components comprising the network can be present in the same pathway, or in different pathways. For example, 1, 2, 3, 4, 5, 6, 7, 8, 9, 10 or more pathways can be included in a network.

The multivariate analysis of the cellular components comprising a signaling network examines numerous conditions of interest simultaneously. Multivariate analysis relies on the ability to sort cellular components or the data associated therewith, during or after the assay is completed. In performing a multivariate assay, the cellular components being detected can be activated, inhibited, or non responsive (i.e., “non-activated”) with respect to an activation event (e.g., phosphorylation or in response to the addition of an agent. An “activated” cellular component is capable of switching from one form to another and exhibits at least one detectable biological, biochemical or physical property or activity, such as the presence of an epitope, presence of a chemical moiety, a conformational change, one or more isoforms, enzymatic activity, etc., in response to an activation event. Examples of suitable activation events include, but are not limited to, a cell signaling event, phosphorylation, cleavage, prenylation, intermolecular clustering, conformational changes, glycosylation, acetylation, cysteinylation, nitrosylation, methylation, ubiquination, sulfation, presence of a particular isoform, and non-covalent binding of inhibitor molecules. A “non-activated” cellular component is a component that lacks or has a diminished level of a detectable biological, biochemical or physical property or activity.

In some embodiments, the activation event comprises the substitution of a phosphate group for a hydroxyl group in the side chain of an amino acid, i.e., phosphorylation. A wide variety of proteins are known that catalyze the phosphorylation of serine, threonine, or tyrosine residues on specific protein substrates. Such proteins are generally termed “kinases.” Substrate proteins that are capable of being phosphorylated are often referred to as phosphoproteins. Once phosphorylated, a substrate protein may have its phosphorylated residue converted back to a hydroxyl group by the action of a protein phosphatase that specifically recognizes the phosphorylated substrate protein. Protein phosphatases catalyze the replacement of phosphate groups by hydroxyl groups on serine, threonine, or tyrosine residues. Through the action of kinases and phosphatases a protein may be reversibly or irreversibly phosphorylated on a multiplicity of residues and its activity may be regulated thereby.

In some embodiments, the activation event comprises the acetylation of histones. Through the activity of various acetylases and deacetylases the DNA binding function of histone proteins is tightly regulated.

In some embodiments, the activation event comprises the cleavage of a cellular component. For example, one form of protein regulation involves proteolytic cleavage of a peptide bond. While random or misdirected proteolytic cleavage may be detrimental to the activity of a protein, many proteins are activated by the action of proteases that recognize and cleave specific peptide bonds. Many proteins derive from precursor proteins, or pro-proteins, which give rise to a mature form of the protein following proteolytic cleavage of specific peptide bonds. Many growth factors are synthesized and processed in this manner, with a mature form of the protein typically possessing a biological activity not exhibited by the precursor form. Many enzymes are also synthesized and processed in this manner, with a mature form of the protein typically being enzymatically active, and the precursor form of the protein being enzymatically inactive. Among the enzymes that are proteolytically activated are serine and cysteine proteases, including cathepsins and caspases, and “zymogens”.

In some embodiments, the activation event comprises the prenylation of a cellular component. By “prenylation” herein is meant the addition of any lipid group to the cellular component. Common examples of prenylation include the addition of farnesyl groups, geranylgeranyl groups, myristoylation and palmitoylation. In general these groups are attached via thioether linkages to the cellular component, although other attachments can be used.

In some embodiments, the activation event comprises a cell signaling event that can be detected as intermolecular clustering of the cellular component. By “clustering” or “multimerization”, and grammatical equivalents used herein, is meant any reversible or irreversible association of one or more signal transduction elements. Clusters can be made up of 2, 3, 4, etc., elements. Clusters of two elements are termed dimers. Clusters of 3 or more elements are generally termed oligomers, with individual numbers of clusters having their own designation; for example, a cluster of 3 elements is a trimer, a cluster of 4 elements is a tetramer, etc.

Clusters can be made up of identical elements or different elements. Clusters of identical elements are termed “homo” clusters, while clusters of different elements are termed “hetero” clusters. Accordingly, a cluster can be a homodimer, as is the case for the β2-adrenergic receptor. Alternatively, a cluster can be a heterodimer, as is the case for GABAB-R. In other embodiments, the cluster is a homotrimer, as in the case of TNFα, or a heterotrimer such the one formed by membrane-bound and soluble CD95 to modulate apoptosis. In further embodiments the cluster is a homo-oligomer, as in the case of thyrotropin releasing hormone receptor, or a hetero-oligomer, as in the case of TGFβ1.

Elements can be activated to cluster through three different mechanisms: a) as membrane bound receptors by binding to ligands (ligands, including both naturally occurring or synthetic ligands), b) as membrane bound receptors by binding to other surface molecules, or c) as intracellular (non-membrane bound) receptors binding to ligands. A variety of membrane bound receptor elements, that cluster by binding to ligands or to other surface molecules, and non-membrane bound receptor elements are taught in copending application Ser. No. 10/898,734, filed Jul. 21, 2004, the disclosure of which is incorporated herein by reference.

In some embodiments, the activation event comprises cleavage, covalent or non-covalent modifications of nucleic acids. For example, many catalytic RNAs, e.g. hammerhead ribozymes, can be designed to have an inactivating leader sequence that deactivates the catalytic activity of the ribozyme until cleavage occurs. An example of a covalent modification is methylation of DNA. Other examples are taught in copending application Ser. No. 10/898,734, filed Jul. 21, 2004, the disclosure of which is incorporated herein by reference.

In some embodiments, cellular components that do not switch from one form to another, and hence exhibit a detectable property in response to an activation event can be detected. Examples of cellular components that are not “activatable” but can be detected using the methods described herein include, but are not limited to, small molecules, carbohydrates, lipids, organic acids, ions, or other naturally occurring or synthetic compounds. As a specific example, activation of cAMP (cyclic adenosine mono-phosphate) can be detected as the presence of cAMP rather than the conversion from non-cyclic AMP to cyclic AMP.

As another specific example, changes in the concentration of a cellular component can be detected. For example, elevated levels of cAMP induce release of PKA, thus, changes in the concentration of cAMP can be detected as an indicator of the activation of PKA. Other examples include, but are not limited to, calcium, mevalonate, thymidine, and glucose. For example, elevated levels of calcium activate calcium-dependent kinases, such as CAMKII, PLCg, and PKC. Elevated levels of mevalonate induce the synthesis of isoprenol derivatives, such as cholesterol, ubiquinone, and dihols, as well as inducing the farnesylation and geranylation of particular proteins, such as Ras, Rho, DNAj, Rap 1. Additionally, very high concentrations of mevalonate induce a negative feedback loop and shut down the activity of HMG-COA reductase, the enzyme that catalyzes mevalonate synthesis. High concentrations of thymidine nucleotides can shut down all of the biosynthetic pathways in a cell. Elevated concentrations of double-thymidine dimers can induce DNA repair pathways, such as the SOS response pathway. Elevated concentrations of glucose induce the production of insulin, which can cause a cell to switch from a metabolic state to a catabolic state characterized by the synthesis and storage of amylose.

In other embodiments, signaling networks associated with the redox state of the cell can be generated by detecting cellular components subject to oxidation/reduction reactions, e.g., gluthathione, thioredoxin, reactive oxygen species (ROS), metals, etc. For example, mitogen-activated protein kinase (MAPK) signaling pathways are reported to be actively involved in transducting oxidative signaling in response to elevated ROS levels.

Examples of other cellular components that are not “activatable” but can be detected using the methods described herein include, but are not limited to, secondary and tertiary RNA structure that can initiate transcriptional arrest, the ratio of mitochondrial housekeeping genes, such as bad/bcl2, and intracellular quantities of mitogenic indicators, such as KI-67, PCNA, histone3-AX, cyclin D, cyclin B, cyclin A and DNA.

In some embodiments, signaling networks are evaluated and characterized using perturbations by exogenously added agents, that ultimately result in alterations in data arc sets and thus can serve to identify decisional arcs. For example, by comparing the data arc set of unperturbed cells and that of the data arc set of cells treated with a drug, the differences, sometimes in the form of decisional arcs, can be determined. In some cases, these agents can be used to derive causal connections between cellular components comprising a signaling network. Generally, the agents modulate one or more of the cellular components comprising a signaling network, resulting in modulation of the data arcs. By “modulate” herein is meant that the agent interacts with the cellular component such that the cellular component switches from one state or form to another. “Agents” in this context include compounds as well as physical parameters. For example, agents can include physical parameters such as heat, cold, radiation (e.g., UV, visible, infrared), pH, salinity, osmolarity, redox potential, electrical gradients, magnetic and x-ray fields. Examples of suitable compounds for use as agents include, but are not limited to, virtually any molecule or compound, including biological molecules (proteins, including peptides, antibodies, cytokines, lipids, nucleic acids, carbohydrates, etc.), non-biological molecules, small molecule drugs, cells, viruses, organic acids, ions, etc. Many of the compounds described above as suitable as “cellular components” can serve as agents. Exemplary drugs include, for example, any compound or composition described in The Merck Index: An Encyclopedia of Chemicals, Drugs, and Biologicals, 13^{th} Ed. (Merck) (Whitehouse Station, N.J.), incorporated herein by reference in its entirety.

Typically, agents can be activators or inhibitors. For example, an activator can be a transcriptional activator, such as DNA binding proteins, which increase the rate of transcription upon binding to DNA. Another example of activators, are positive modulators of allosteric enzymes that upon binding mediate a conformation change between an inactive to an active form. Positive modulators include enzyme substrates, cofactors, natural or synthetic, metabolically active or inactive steroid or steroid analogues. Agents that can act as inhibitors, generally interact with a cellular component that such the cellular component is switched from an active form to an inactive form. Examples of suitable inhibitors include protein kinase inhibitors, statin molecules, HMG-COA reductase inhibitors, FLT3 kinase inhibitors, and transcriptional inhibitors.

Other examples of agents capable of impacting cellular signaling networks, including potentiators, are taught in co-pending application Ser. No. 10/898,734, filed Jul. 21, 2004, the disclosure of which is incorporated herein by reference.

One or more agents can be used to generate independent perturbation events to for example derive causal connections between cellular comprising a signaling network. For example, one agent can be used. As another example, two, three, four, five, six, seven, eight, nine, ten, or more agents can be used. In yet another example, between 10 to 100 agents can be used, provided that the perturbation events induced by the different agents can be detected using the methods described herein.

The agents can all have the same effect, some of the agents can have the same effect and others can have a different effect, or all the agents can have a different effect. For example, a combination of inhibitors and activators can be used to generate multiple independent perturbation events. The combinations can comprise an equal number of activators and inhibitors, or an unequal number of activators to inhibitors. For example, two activators and two inhibitors can be used. As another example, two activators and five inhibitors are used. Thus, any number and combination of activators and inhibitors can be used, provided that the effects generated by each, can be detected and correlations between the different cellular components made using the methods described herein. Disease states can also be characterized. A first set of arcs for a set of cellular components from an individual cell exhibiting said disease state is provided. A second set of arcs is then provided for a set of cellular components from an individual cell not exhibiting the disease state. The first and second sets are then compared to determine one or more decisional arcs indicative of said disease state.

Diseases can be diagnosed or prognosed using the methods disclosed herein. For example, a set of one or more decisional arcs indicative of the presence or absence of the disease state is provided. A model of cell networks in each cell obtained from a subject are detected obtain a set of one or more arcs. The arcs are then compared a set of decisional arcs to diagnose the disease state in the subject. Alternatively, the procedure can be adapted to prognose a disease state in the subject.

In some embodiments, different cell types can be used in place of agents to generate cell signaling networks. Typically, the different cell types will comprise two, three, four, five, or more populations of cells. By “population” herein is meant a group of cells isolated from a specific organ, tissue or individual. The cell populations can be isolated from the same organ, tissue or individual, or from different organs, tissues, or individuals. For example, in some embodiments, the cell populations can be isolated from one or more individuals and comprise cell types implicated in a wide variety of disease conditions, even while in a non-diseased state. Suitable eukaryotic cell types include, but are not limited to, tumor cells of all types (including primary tumor cells, melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as hemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Disease states include but are not limited to diseases associated with any of the listed cell types, including cancer, autoimmune diseases (including rheumatoid arthritis, multiple schlerosis, and lupis), inflammation, heart conditions, allergies and asthma, and depression and other neurological disorders.

As another specific example, the cell populations can be isolated from the same organ or different organs to generate signaling networks involved in homeostasis. Additionally, differences between specific primary sell types and cell subpopulations can be used to generate signaling networks using the methods described herein. In some embodiments, the methods can be extended to include whole animal studies, such as whole body fluorescence imaging of phosphorylation states in Caenorhabditis elegans or Drosophila larva.

The cellular components comprising a cell signaling network can be detected using a variety of different methods. For example, probes can be designed that detect a specific isoform of a protein, such as one of the three isoforms of TGF-β. As another example, probes can also be designed to detect epitopes that are exposed as result of a conformational change in cellular component. In another example, probes can be designed that detect a modification of a cellular component, such as caused by the addition or removal of a chemical group. In other examples, probes can be designed to detect cellular components, that do not undergo a change in form or state due to a perturbation event, phospholipids, organic acids, ions, etc. Additional examples of methods for detecting cellular components are taught in co-pending application Ser. No. 10/898,734, filed Jul. 21, 2004, the disclosure of which is incorporated herein by reference by its entirety.

Generally, a set of probes is used to detect the presence or absence of one or more cellular components. A set of probes can comprise a single probe or more than one probe. For example, in some embodiments, a set can comprise 1, 2, 3, 4, 5, 6, 7, 8, 9, 10, 11, 12, 13, 14, 15, 16, 17, 18, 19, 20, or more probes. The number of probes in a set can be selected based upon a number of factors, such as the number of unique cellular components present in an assay, or on the number of different detectable labels available for a given assay format.

Virtually any molecule can be used as probe to detect one or more of the cellular component described herein. Suitable probes include, but are not limited to, proteins, peptides, nucleic acids, antibodies, organic compounds, small molecules, and carbohydrates. Additional examples of binding elements suitable for use as probes in the methods described herein are taught in co-pending application Ser. No. 10/898,734, filed Jul. 21, 2004, the disclosure of which is incorporated herein by reference by its entirety.

In some embodiments, antibodies can be used as probes. By “antibody” herein is meant a protein consisting of one or more polypeptides substantially encoded by all or part of the recognized immunoglobulin genes. The recognized immunoglobulin genes, for example in humans, include the kappa (k), lambda (I), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. The term “antibody” includes antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. Particularly preferred are full length antibodies that comprise Fc variants as described herein. The term “antibody” comprises monoclonal and polyclonal antibodies. Antibodies can be antagonists, agonists, neutralizing, inhibitory, or stimulatory.

The antibodies can be nonhuman, chimeric, humanized, or fully human. For a description of the concepts of chimeric and humanized antibodies see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see for example U.S. Pat. No. 4,816,567). In a preferred embodiment, the antibodies of the present invention are humanized. By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). The humanized antibody optimally also will comprise at least a portion of an immunoglobulin constant region, typically that of a human immunoglobulin, and thus will typically comprise a human Fc region. Methods for humanizing non-human antibodies are well known in the art, and can be essentially performed following the method of Winter and co-workers (Jones et al., 1986, Nature 321:522-525; Riechmann et al., 1988, Nature 332:323-329; Verhoeyen et al., 1988, Science, 239:1534-1536). Additional examples of humanized murine monoclonal antibodies are also known in the art, for example antibodies binding human protein C (O'Connor et al., 1998, Protein Eng 11:321-8), interleukin 2 receptor (Queen et al., 1989, Proc Natl Acad Sci, USA 86:10029-33), and human epidermal growth factor receptor 2 (Carter et al., 1992, Proc Natl Acad Sci USA 89:4285-9). In an alternate embodiment, the antibodies of the present invention may be fully human, that is the sequences of the antibodies are completely or substantially human. A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

Included within the definition of “antibody” are aglycosylated antibodies. By “aglycosylated antibody” herein is meant an antibody that lacks a carbohydrate attached at position 297 of the Fc region, wherein numbering is according to the EU system as in Kabat. The aglycosylated antibody may be a deglycosylated antibody, which is an antibody for which the Fc carbohydrate has been removed, for example chemically or enzymatically. Alternatively, the aglycosylated antibody may be a nonglycosylated or unglycosylated antibody, that is an antibody that was expressed without Fc carbohydrate, for example by mutation of one or residues that encode the glycosylation pattern or by expression in an organism that does not attach carbohydrates to proteins, for example bacteria.

Also included within the definition of “antibody” are full-length antibodies that contain an Fc variant portion. By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, Cg1, Cg2, and Cg3. In some mammals, for example in camels and llamas, IgG antibodies may consist of only two heavy chains, each heavy chain comprising a variable domain attached to the Fc region. By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

Antibodies can be designed to bind a specific antigen or epitope associated with a specific activated state of a cellular component. For example, antibodies can be designed that recognize a transition state for a known enzyme, a specific isoform of a protein, or the presence or absence of a covalent or non-covalent modification (see, e.g., co-pending application Ser. No. 10/898,734, filed Jul. 21, 2004, the disclosure of which is incorporated herein by reference by its entirety).

The probes typically comprise a reporter or a signal label capable of producing a detectable signal when the labeled probe binds to a cellular component. A labeled probe can comprise a label that is attached directly to the probe and is detectable or produces a detectable signal. The labels may be attached to the labeled probes at virtually any position. For example, if the probe is a nucleic acid, the labels may be attached to a terminus, to a terminal or internal nucleobase or to the backbone. If the probe is an antibody, the label can be attached to any amino acid residue, provided that the label does not interfere with the binding of the probe to a cellular component. Although the type of label is not critical to success, the labels used should produce detectable signals. The various detectable labels of a set of probes should be different and distinguishable. By “distinguishable” we mean that the labels should be spectrally resolvable from one another.

The number of labels used in the probe sets can depend on the number of spectrally resolvable labels available and the labeling method. For example, from 1 to 7 fluorophores can be used as labels for the probes. In contrast, if quantum dots are used to label the probes, the number of spectrally resolvable labels can vary from 1 to 24, or more than 24 depending on the assay conditions.

The labeled probe can comprise a label that is a fluorophore. Non-limiting examples of fluorophores suitable for labeling probes used in the methods described herein include Spectrum-Orange™, Spectrum-Green™, Spectrum-Aqua™, Spectrum-Red™, Spectrum-Blue™, Spectrum-Gold™, fluorescein isothiocyanate, rhodamine, and FluroRed™, 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.) or the Alexa dye series (Molecular Probes, Eugene, Oreg.).

Additional labels that can be detect via fluorescent properties, including, but not limited to, Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680, Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.) are taught in co-pending application Ser. No. 10/898,734, filed Jul. 21, 2004, the disclosure of which is incorporated herein by reference.

In some embodiments, the label can be a microsphere comprising a spectral code, commonly referred to in the art as a “quantum dot” (see U.S. Pat. No. 6,500,622, the disclosure of which is incorporated herein by reference). The spectral code can comprise one or more semiconductor nanocrystals, having at least one different fluorescent characteristic, for example excitation wavelength, emission wavelength, emission intensity, etc. By attaching the probes to quantum dots having a range of distinguishable spectra allows for the simultaneous analysis of more cellular components than is currently possible using existing fluorophores. For example, 12 or more spectrally resolvable labels can be used in a single assay. Such label formats are particularly well suited for use in multiplex assays because of the tremendous diversity of different, distinguishable; detectable labels.

The number of spectrally resolvable labels used in a single assay can also be increased by using combinatorial or ratiometric labeling. In combinatorial labeling, the number of all possible combinations is described by the formula X=2ⁿ−1, where n refers to the number of labels used. Using three fluorescent-labeled nucleotides (FITC-dUTP, Cy3-dUTP and AMCA-dUTP), seven different DNA probes can be labeled and simultaneously identified after hybridization, based on color combinations. For example, a DNA probe labeled with FITC will fluoresce green, another one labeled with AMCA will fluoresce blue, whereas a third one labeled with FITC and AMCA will fluoresce cyan. Similarly, combing probes in which one probe is labeled red and the other with green yields a yellow signal, the combination of a blue and a red labeled probes yields a magenta signal, whereas the combination of probes in which one probe is labeled with FITC-green, another is labeled with AMCA-blue and a third is labeled with Cy3-orange/red fluoresces “white”.

If ratio labeling is used, in theory many targets can be distinguished with a few labels. With ratio labeling, a mixture of probes is used wherein each probe is labeled with a resolvable label. The amount of each probe used in the mixture is at a set ratio to one another. Each target is distinguished by possessing different ratios of the colors used. For example, using two labels, red and green, a first target can be detected using only red labeled probes (i.e. target appears red), a second target can be detected using only green labeled probes (i.e. target appears green), a third target can be detected using a mixture of a red labeled probes and green labeled probes at a ratio of 75:25, such that the third target is distinguished from the first target based on the shade of red observed (i.e., the third target will be a less intense shade of red), a fourth target can be detected using a mixture of a red labeled probes and green labeled probes at a ratio of 65:35, such that the fourth target is distinguished from the first and third targets, again based on the shade of red observed (i.e., the fourth target appears orange), a fifth target can be detected using a mixture of a red labeled probes and green labeled probes at a ratio of 50:50, such that the fifth target is appears yellow, and so forth. Computer software is often required to sufficiently distinguish the different ratios.

The use of multicolor, multiparameter flow cytometry requires primary conjugated antibodies at defined fluorophores to protein (“FTP”) ratios. It is generally not sufficient to give a range of FTP ratios, but rather it is necessary to quantitate the final product thoroughly as FTP ratios differing in 2 molecules can represent significant decreases in phospho-epitope staining. It is also important to note that each fluorophore's optimal FTP is unique and can differ amongst antibody clones to phospho-epitopes.

In some embodiments, the optimal ratio for any protein fluorophore (i.e. PE, APC, PE-TANDEM CONJUGATES (PE-TR, PE-Cy5, PE-CY5.5, PE-CY7, PE-Alexa colors (PE-AX610, PE-AX647, PE-680, PE-AX700, PE-AX750), APC-TANDEM CONJUGATES APC-AX680, APC-AX700, APC-AX750, APC-CY5.5, APC-CY7), GFP, BFP, CFP, DSRED, and all the derivates of the algae proteins including the phycobilliproteins is 1:1 (one ab to one protein dye).

In additional embodiments, the FTP ratio is 1-6 for internal stains; for AX488 the FTP is preferably 2-5 and more preferably 4; for AX546 the FTP ratio is preferably 2-6 and more preferably 2; for AX594 the FTP ratio is preferably 2-4; for AX633 the FTP is preferably 1-3; for AX647 the FTP ratio is preferably 1-4 and more preferably 2. For AX405, AX430, AX555, AX568, AX680, AX700, AX750 the FTP ratio is preferably 2-5.

Alternatively, detection systems based on FRET, discussed in detail in co-pending application Ser. No. 10/898,734, filed Jul. 21, 2004, (the disclosure of which is incorporated by reference in its entirety) can be used in the methods described herein.

A number of other labels, such “label enzymes”, “secondary labels”, radioisotope and methods for detecting these labels are taught in co-pending application Ser. No. 10/898,734, filed Jul. 21, 2004, the disclosure of which is incorporated by reference in its entirety.

Any prokaryotic or eukaryotic cell can be used in the methods described herein. Suitable prokaryotic cells include, but are not limited to, bacteria such as E. coli, various Bacillus species, and the extremophile bacteria such as thermophiles, etc.

Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tilapia, tuna, carp, flounder, halibut, swordfish, cod and zebra fish. Suitable bird cells include, but are not limited to, those of chickens, ducks, quail, pheasants and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes. As discussed above, suitable cells also include cell types implicated in a wide variety of disease conditions.

Suitable cells also include known research cells, including, but not limited to, Jurkat T cells, NIH3T3 cells, CHO, COS, etc. Suitable cells also include primary cells obtained from a subject. See the ATCC cell line catalog, hereby expressly incorporated by reference.

A number of different methods can be used to detect the cellular components comprising a signaling network. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above.

Cellular components may be detected by any methods in the art. In some embodiments, the methods comprise detecting cellular components comprising a labeled probe in individual cells using FACS. Different types of fluorescent monitoring systems, e.g., FACS systems, can be used to detect labeled cellular components. For example, FACS systems dedicated to high throughput screening, e.g., 96 well or greater microtiter plates, can be used. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

In some embodiments, flow cytometry is used to detect fluorescence. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remacle et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating steps can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods described herein (see e.g., WO99/54494, filed Apr. 16, 1999; U.S.S.N. 20010006787, filed Jul. 5, 2001, each expressly incorporated herein by reference). As a specific example, a FACS cell sorter (e.g. a FACSVantage™ Cell Sorter, Becton Dickinson Immunocytometry Systems, San Jose, Calif.) can be used to sort and collect cells based on whether or not a labeled probe has bound a cellular constituent.

Additional methods for detecting cellular components using FACS are described in co-pending application Ser. No. 10/898,734, filed Jul. 21, 2004, the disclosure of which is incorporated herein by reference in its entirety and in the Examples.

Other methods for detecting cellular components, such as the used of arrays and confocal microscopy are taught in co-pending application Ser. No. 10/898,734, filed Jul. 21, 2004, the disclosure of which is incorporated herein by reference in its entirety.

Protocols for detecting cellular components using FACS are taught in co-pending application Ser. No. 10/898,734, filed Jul. 21, 2004, the disclosure of which is incorporated herein by reference in its entirety, and in the Examples.

Bayesian networks can be used to analyze the multiple measurements of cellular components obtained using multicolor flow cytometry. Bayesian networks (Pearl, J. (1988), supra) provide a compact graphical representation of multivariate joint probability distributions. This representation consists of a directed acyclic graph whose nodes correspond to random variables, each representing the measured levels of a biomolecule in the dataset. An arc expresses statistical dependence of the downstream variable on the upstream (parent) variable. In certain cases, these statistical dependencies can be interpreted as causal influences from the parent upon the downstream variable (molecule) (Pearl, J. (2000) Causality: Models, Reasoning, and Inference (Cambridge University Press). The Bayesian network associates with each variable Xi, a probability distribution conditioned on its parents in the graph (Paᵢ). Intuitively, the values of the parents directly influence the value for Xi. The graph structure represents the dependency assumptions that each variable is independent of its non-descendents, given its parents in the graph; thus the joint distribution can be decomposed into the following product form:
P(X₁, . . . , Xₙ)=ΠP(Xᵢ|Paᵢ)

The goal of Bayesian network inference is to search among possible graphs and select a graph or graphs that best describe the dependency relationships observed in the empirical data. If a score based approach is used, a statistically motivated scoring function is introduced that evaluates each network with respect to the data, and searches for the highest scoring network. Since the datasets generated using the methods described herein contain conditions that directly manipulate the levels of the measured biomolecules (i.e., cellular components), an adaptation of the standard Bayesian scoring metric (Heckerman, D. (1995) in Microsoft Research, Vol. MSR-TR-95-06) is used that explicitly models these interventions as described in (Pe'er, D., Regev, A., Elidan, G. & Friedman, N. (2001) Bioinformatics 17 Suppl 1, S215-24, Yoo, C. a. C. G. F. (1999) in Uncertainty in Artificial Intelligence, pp.116-125). This score rewards relatively simple models (i.e. few arcs), that were likely to have generated the data, i.e., whose underlying distribution is close to the empirical distribution of the data. Once the score is specified and the data is given, network inference amounts to finding the structure that maximizes the score. The number of possible graph structures is super-exponential in the number of variables (measured biomolecules) and therefore the size of the search space prohibits an exhaustive search. Thus, a heuristic simulated annealing search is used. A search space is defined where each state is a possible network structure and a set of operators is defined: addition, deletion or reversal of a single arc, that transform from one structure to another. The search is started with an initial random structure and this space is traversed using the operators to search for high scoring networks. At each step in the search procedure, a random operator is used to change the graph, the resulting structure is rescored and the change is incorporated if it yields an improvement in the score. To avoid local maxima, occasionally a change is incorporated even if it decreases the score. This procedure is iterated to find high-scoring graphs.

This process can be initialized with different random graphs and repeated many times (e.g., 500 times), to explore different regions of the search space. Typically, many of the resulting models explain the data almost equally well among themselves. To gain statistical robustness in the inference obtained, instead of relying on a single high scoring structure, model averaging can be performed on the compendia of high scoring networks (Pe'er, D., Regev, A., Elidan, G. & Friedman, N. (2001) Bioinformatics 17 Suppl 1, S215-24). This results in an averaged network, consisting of common features (arcs), on which most of the high scoring network structures agree. The final inferred networks consists of arcs of confidence 85% or greater.

In some embodiments, correlation connections between different cellular components can be made using a Bonferroni corrected p value.

FIGS. 1B and C illustrate the application of the Bayesian network inference algorithm to a hypothetical signaling network. FIG. 1B (upper panel, ‘α’ diagram) depicts an example of a Bayesian network representing 4 different hypothetical biomolecules (i.e., cellular components). A directed arc from X to Y is interpreted as a causal influence from X onto Y; e.g., X is Y's parent in the network. In the case that X activates Y, correlation in levels of the two protein activities as measured by flow cytometry are expected and observed (see simulated data in FIG. 1C panel i). To assign causality to the relationship, events that directly perturb the states of the measured molecules are employed (see FIG. 1C panel ii). For example, inhibition of molecule X leads to inhibition of both X and Y, whereas inhibition of molecule Y only leads to inhibition of Y, thus, X is inferred to be upstream of X as per the original diagram in FIG. 1B (upper panel ‘a’). Moreover, since flow cytometry can measure multiple molecules within each cell, it is possible to identify complex causal influence relationships involving multiple proteins. Consider the signaling cascade from X onto Y onto Z (FIG. 1B, upper panel) where correlation exists between the measured activities of each pair, including between X and Z (FIG. 1C panel iv). Bayesian network inference chooses the most concise model, automatically excluding arcs based on dependencies already explained by the model. Thus, despite the correlation between them, the arc between X and Z is omitted, since the X-Y and the Y-Z relationships (FIG. 1C panel i and iii, respectively) explain the X-Z correlation. Similarly, since Z and W are both activated by their common cause Y, their activities are expected to be correlated, but no arc appears between them because the respective arcs from Y mediate this dependency (dataset not shown). Finally, consider a scenario in which molecule Y was not measured. In this scenario, the statistical correlation between the observed activities of X and Z does not depend on observing Y, therefore, their correlation will still be detected. Since Y's activities are unobserved, there is no molecule in the data that can explain this dependency, thus, an indirect arc occurs from X onto Z (FIG. 1B, lower panel, ‘β’ diagram).

FIGS. 3A and 3B illustrate the application of the Bayesian network inference algorithm to datasets obtained using flow cytometry measurements of 11 phosphoproteins and phospholipids (Raf-259, Erk1/2-T202/T204, p38-T180/Y182, Jnk-T183/Y185, Akt-S473, Mek1/2-S217/S221, PKA substrates, PKC-S660, Plcg-Y783, PIP2, PIP3) in human primary naive CD4+ T cells. Agents used to activate or inhibit the 11 phosphoproteins and phospholipids are shown below in Example 1. The resulting de novo inferred causal network model is shown in FIG. 3A, with 17 high-confidence causal arcs derived between various components.

To evaluate the validity of this model, a comparison was made of the model arcs—and absent potential arcs—with previous literature reports. Of the 17 arcs in the model shown in FIG. 3A, 14 are classified as expected, 16 are found in the literature (expected or reported), 1 is not previously reported (unexplained), and 4 are classically expected, but were missed (FIG. 3A). The probable paths of influence corresponding to model arcs are shown below in Table 1.

TABLE 1

| | | | Cate- |
|---|---|---|---|
| Connection | Influence path | Type | gory^{1,2} |
| PKC→ Raf | PKC→ Ras→ Raf_{S259} | indirect | E |
| PKC→ Mek | PKC→Raf_{S497/S499} → Mek | indirect | E |
| PKC→ Jnk | PKC→→ MKKS→ Jnk | indirect | E |
| PKC→ p38 | PKC→→ MKKs→ p38 | indirect | E |
| PKC→ PKA | PKC → cAMP → PKA | indirect | R |
| PKA→ Raf | PKA → Raf_{S259} | direct | E |
| PKA→ Mek | PKA→ Raf_{S621}→ Mek | indirect | E |
| PKA→ Erk | Unknown | | U |
| PKA→ Jnk | PKA→ → MKKs→ Jnk | indirect | E |
| PKA→ p38 | PKA→ → MKKS→ Jnk | indirect | E |
| Raf→ Mek | direct phosphorylation | direct | E |
| PKA→ Akt | PKA→ CaMKK→ Akt_{T308}→ Akt_{S473} | indirect | E |
| Mek → Erk | direct phosphorylation | direct | E |
| Plcγ→ PIP2 | direct phosphorylation | direct | E |
| Plcγ→ PIP3 | direct phosphorylation | reversed | E |
| PIP3→ PIP2 | Precursor | | E |
| Erk→ Akt | direct or indirect | | R |
| ¹E = expected, U = unexplained, R = reported. | | | |
| ²References used for comparisons: M. P. Carroll, W. S. May, J Biol Chem 269, 1249 (Jan. 14, 1994), R. Marais, Y. Light, H. F. Paterson, C. J. Marshall, Embo J 14, 3136 (Jul. 3, 1995), R. Marais et al., Science 280, 109 (Apr. 3, 1998), W. M. Zhang, T. M. Wong, Am J Physiol 274, C82 (January, 1998), R. Fukuda, B. Kelly, G. L. Semenza, Cancer Res 63, 2330 (May 1, 2003), P. A. Steffen M, Aach J, D'haeseleer P, Church G., BMC Bioinformatics. 1, 34 (Nov. 1, 2002), Y. B. Kelley BP, | | | |
| # Lewitter F, Sharan R, Stockwell BR, Ideker T., Nucleic Acids Res. 32, W83 (Jul. 1, 2004), K. M. Nir Friedman, and Stuart Russell, paper presented at the Uncertainty in Artificial Intelligence, Madison, Wisconsin, July 1998, J. D. G. Irene M. Ong, and David Page, Bioinformatics 18, S241 (2002), M. Roederer, J. M. Brenchley, M. R. Betts, S. C. De Rosa, Clin Immunol 110, 199 (March, 2004), and A. Perfetto, Chattopadhyay, P., Roederer, M., Nature Reviews Immunology 4, 648 (2004). | | | |

For a complete discussion of the above model, see Examples.

Traditional understanding of pathway structures as collated from diverse model cell types and organisms demonstrates the essential congruity of basic signaling networks, but does not easily reveal the subtle differences that exist in different primary cell subtypes. Application of Bayesian Network Analysis to sets of molecules, cell types, disease states and interventions (e.g., siRNA and dominant negative screens, or pharmaceutical agents) can be used to develop signaling networks in a single experimental/computational approach, especially with respect to complex, nonlinear cross-talk between pathways. Application of this approach during biochemical interrogation of cellular subset-specific signaling networks in the course of disease state or in the presence of pharmaceutical agents can potentially provide important mechanistic information of clinical relevance. For example, this method can be used to identify sets of signaling molecules that explain differences between responses to chemotherapy in patients with cancer (Marais, R., Light, Y., Mason, C., Paterson, H., Olson, M. F. & Marshall, C. J. (1998) Science 280, 109-12).

All publications, patent applications, and similar-materials mentioned herein are hereby incorporated by reference in their entirety for any purpose. In the event that one or more of the incorporated materials differs from or contradicts this application, including but not limited to defined terms, term usage, described techniques, or the like, this application controls.

The following Examples are illustrative of the disclosed compositions and methods, and are not intended to limit the scope of the various embodiments described herein. Without departing from the spirit and scope of the present teachings, various changes and modifications of the present teachings will be clear to one skilled in the art and can be made to adapt the present teachings to various uses and conditions. Thus, other embodiments are encompassed.

### A. EXAMPLES

b. Modeling of a Cell Signaling Network Using the Bayesian Network Inference Algorithm

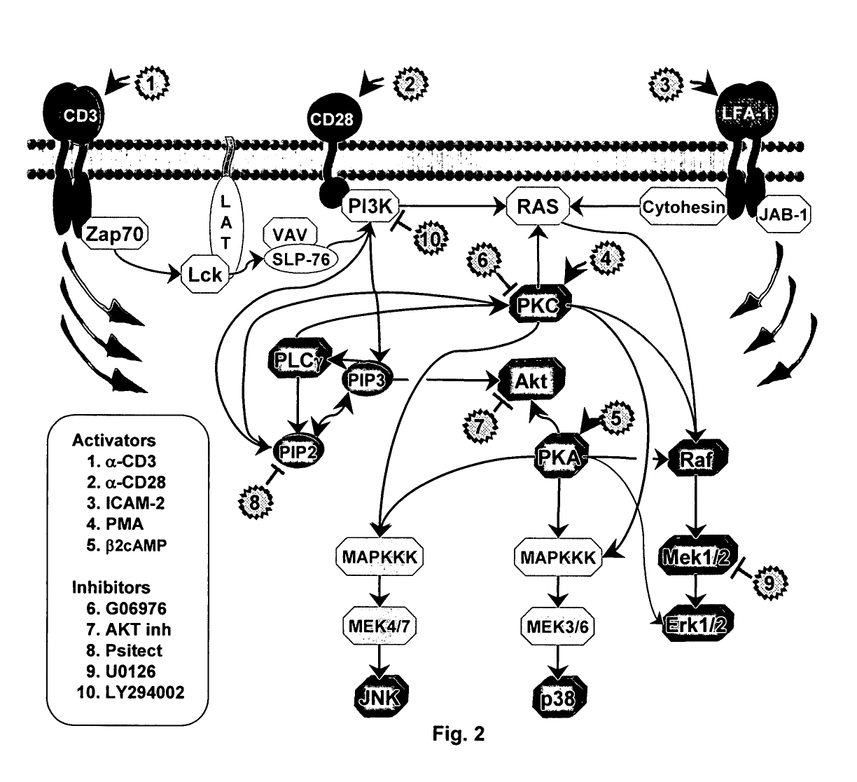

We applied Bayesian network analysis to multivariate flow cytometry data. Data were collected after a series of stimulatory cues (e.g., activators) and inhibitory interventions (see Table 2), with cell reactions stopped at 15 minutes post-stimulation by fixation, to profile the effects of each condition on the intracellular signaling networks of human primary naive CD4+ T cells, downstream of CD3, CD28, and LFA-1 activation (see FIG. 2 for a currently accepted consensus network).

TABLE 2

| Perturbations | Reagent | Reagent Class |
|---|---|---|
| anti-CD3 + anti- | anti-CD3/CD28 | General perturbation: Activates |
| CD28 | | T cells and induces proliferation |
| | | and cytokine production. |
| | | induces signaling through the |
| | | TCR, activated ZAP70, Lck, |
| | | PLCγ, Raf, Mek, ERK, PKC. |
| | | TCR signaling that converge on |
| | | transcription factors NFKB, |
| | | NFAT, and AP-1 to initiate IL-2 |
| | | transcription. |
| anti-CD3/CD28 + ICAM-2 | ICAM-2 | General perturbation: Induces |
| | | LFA-1 signaling and contributes |
| | | to CD3/CD28 signaling that |
| | | converge on AP-1 and NFAT |
| | | transcriptional activity. |
| anti-CD3/CD28 + U0126 | β2cAMP | Specific perturbation: cAMP |
| | | analog that activates PKA. |
| | | PKA can regulate NFAT |
| | | activation and T cell |
| | | Commitment processes. |
| anti-CD/3CD28 + AKT- | AKT-inhibitor | Specific perturbation: Binds |
| inhibitor | | inositol pleckstrin domain of |
| | | AKT and blocks AKT |
| | | translocation to the membrane |
| | | where normally AKT it becomes |
| | | phosphorylated and active. |
| | | (IC₅₀ = 5 μM). Inhibition of AKT |
| | | and phosphorylation of AKT |
| | | substrates needed to enhance |
| | | cell survival. |
| anti-CD/3CD28 + G06976 | U0126 | Specific perturbation: Inhibits |
| | | MEK1 (IC₅₀ = 72 nm) and MEK2 |
| | | (IC₅₀ = 58 nm) in a |
| | | noncompetitive manner (ATP |
| | | and ERK substrates). Inhibits |
| | | activation of ERK, arresting T |
| | | cell proliferation and cytokines |
| | | synthesis. |
| anti-CD3/CD28 + Psitectorigenin | PMA | Specific perturbation: Phorbol |
| | | myristate acetate that activates |
| | | PKC, initiates some aspects of |
| | | T cell activation |
| anti-CD3/CD28 + LY294002 | G06976 | Specific perturbation: Inhibits |
| | | PKC isozymes (IC₅₀ < 8 nM). |
| | | Inhibits PKC, arrests T cell |
| | | activation. |
| PMA | Psitectorigenin | Specific perturbation: Inhibits |
| | | phosphoinositide hydrolysis. |
| | | Inhibits PIP2 production, |
| | | disrupts phosphoinositol |
| | | turnover. |
| β2cAMP | LY294002 | Specific perturbation: PI3K |
| | | inhibitor. Inhibits PI3K and |
| | | subsequent activation of AKT. |

Flow cytometry measurements of the following 11 phosphorylated proteins and phospholipids were made: Raf phosphorylated at position S259, mitogen activated protein kinase Erk1 and Erk2 phosphorylated at T202 and Y204, p38 MAPK phosphorylated at T180 and Y182, JNK phosphorylated at T183 and Y185, AKT phosphorylated at S473, Mek 1 and Mek2 phosphorylated at S217 and S221 (both isoforms of the protein are recognized by the same antibody), phosphorylation of PKA substrates (CREB, PKA, CAMKII, CASPASE 10, CASPASE 2) containing a consensus phosphorylation motif, phosphorylation of PLCg on Y783, phosphorylation of PKC on S660, and phosphor-inositol 4,5 bisphosphate [PIP2] and phosphoinositol 3,4,5 triphosphate [PIP3] (see Table 3, Materials and Methods, and Wayman G A, T. H., Soderling T R. (1997) J Biol Chem 26,16073-6).

TABLE 3

| Measured | |
|---|---|
| Molecule | Antibody specificity |
| Raf | Phosphorylation at Serine 259 |
| ERK1 and | Phosphorylation at Threonine 202 and Tyrosine 204 |
| ERK2 | |
| p38 | Phosphorylation at Threonine 180 and Tyrosine 182 |
| JNK | Phosphorylation at Threonine 183 and Tyrosine 185 |
| AKT | Phosphorylation at Serine 473 |
| MEK 1 and | Phosphorylation at Serine 217 and Serine 221 |
| MEK 2 | |
| PKA | Detects proteins and peptides containing a phospho-Ser/Thr |
| substrates | residue with arginine at the −3 position |
| PKC | Detects phosphorylated PKC alpha, beta I, beta II, delta, |
| | epsilon, eta and theta isoforms only at carboxy-terminal |
| | residue homologous to seine 660 of PKC beta II. |
| PLCγ | Phosphorylation at Tyrosine 783 |
| PIP2 | Detects phosphoinositol 4,5 bisphosphate |
| PIP3 | Detects phosphoinositol 3,4,5 triphosphate |

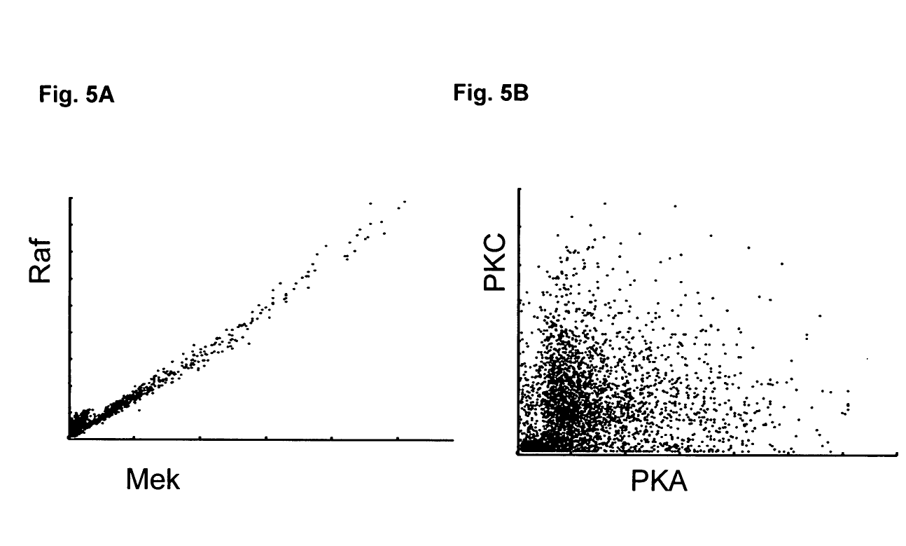

Each independent sample in this dataset consists of quantitative amounts of each of the 11 phosphorylated molecules, simultaneously measured from single cells (see *Appendix 1, Datasets). For purposes of illustration, examples of actual FACS data plotted in prospective co-relationship form are shown in FIG. 5. In most cases, this reflects the activation state of the kinases monitored, or in the cases of PIP3 and PIP2 the levels of these secondary messenger molecules in primary cells, under the condition measured. Nine stimulatory or inhibitory interventional conditions were used (see Table 2, Materials and Methods, and Wayman G A, T. H., Soderling T R. (1997) J Biol Chem 26, 16073-6). The complete datasets were analyzed with the Bayesian network structure inference algorithm (Pe'er, D., Regev, A., Elidan, G. & Friedman, N. (2001) Bioinformatics 17 Suppl 1, S215-24, Marais, R., Light, Y., Paterson, H. F. & Marshall, C. J. (1995) Embo J 14, 3136-45). The resulting de novo causal network model was inferred (FIG. 3A) with 17 high-confidence causal arcs between various components.

To evaluate the validity of this model, we compared the model arcs—and absent potential arcs-with those described in the literature. Arcs were categorized as: [i] ‘expected,’ for connections well-established in the literature, that have been demonstrated under numerous conditions in multiple model systems; [ii] ‘reported,’ for connections that are not well known, but for which we were able to find at least one literature citation; [iii] ‘unexplained,’ indicates that though the arc was inferred from our model, no previous literature reports were found; and [iv] ‘missing’ indicates an expected connection that our Bayesian network analysis failed to find. As used herein, an ‘unknown’ arc is synonymous with an ‘unexplained’ arc. Of the 17 arcs in our model, 14 were expected, 16 were either expected or reported, 1 was not previously reported (unexplained), and 4 were missed (FIG. 3A) (Jaumot, M. & Hancock, J. F. (2001) Oncogene 20, 3949-58, Marshall, C. J. (1994) Curr Opin Genet Dev 4, 82-9, Carroll, M. P. & May, W. S. (1994) J Biol Chem 269, 1249-56, Clerk, A., Pham, F. H., Fuller, S. J., Sahai, E., Aktories, K., Marais, R., Marshall, C. & Sugden, P. H. (2001) Mol Cell Biol 21, 1173-84, and Zhang, W. M. & Wong, T. M. (1998) Am J Physiol 274, C82-7). Table 1 enumerates the probable paths of influence corresponding to model arcs determined by surveying published reports.

Several of the known connections from the model are direct enzyme-substrate relationships (FIG. 3B): PKA to Raf, Raf to Mek, Mek to Erk, Plcg to PIP2; and, one a relationship of recruitment leading to phosphorylation Plcg to PIP3. In almost all cases, the direction of causal influence was correctly inferred (an exception was Plcg to PIP3, in which case the arc was inferred in the reverse direction). All the influences are contained within one global model, thus the causal direction of arcs is often compelled so that these are consistent with other components in the model. These global constraints allowed detection of causal influences from molecules that were not perturbed in the assay. For instance, although Raf was not perturbed in any of the measured conditions, the method correctly inferred a directed arc from Raf to Mek—as expected for the well characterized Raf-Mek-Erk signal transduction pathway. In some cases, the influence of one molecule on another is mediated by intermediate molecules that were not measured in the dataset. In the results, these indirect connections were detected as well (FIG. 3B, panel b). For example, the influence of PKA and PKC on the MAPKs p38 and Jnk likely proceeded via their respective (unmeasured) MAPK kinase kinases. Thus, unlike some other approaches used to elucidate signaling networks (for example, protein-protein interaction maps (Dhillon, A. S., Pollock, C., Steen, H., Shaw, P. E., Mischak, H. & Kolch, W. (2002) Mol Cell Biol 22, 3237-46; Mischak, H., Seitz, T., Janosch, P., Eulitz, M., Steen, H., Schellerer, M., Philipp, A. & Kolch, W. (1996) Mol Cell Biol 16, 5409-18)) that provide static biochemical association maps with no causal links, the Bayesian network method can detect both direct and indirect causal connections and therefore provide a more contextual picture of the signaling network.

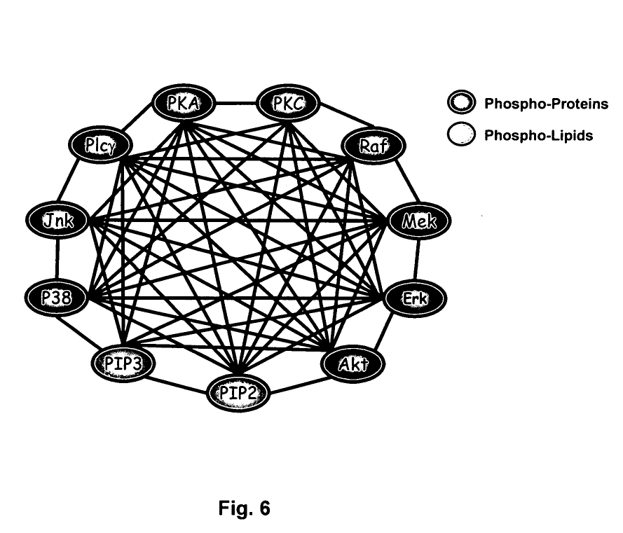

Another important feature of the model is the ability to dismiss connections that are already explained by other network arcs (see, e.g., FIG. 3B panel c). This is seen in the Raf-Mek-Erk cascade. Erk, also known as p44/42, is downstream of Raf and therefore dependent on Raf, yet no arc appears from Raf to Erk, as the connection from Raf to Mek, and from Mek to Erk, explains the dependence of Erk on Raf. Thus, an indirect arc should appear only when one or more intermediate molecules is not present in the dataset, otherwise the connection will proceed via this molecule. The intervening molecule may also be a shared parent. For example, phosphorylation status of p38 and Jnk are correlated (FIG. 6), yet they are not directly connected, as their shared parents (PKC and PKA) mediate the dependence between them. Although the model does not indicate whether an arc represents a direct or indirect influence, it is unlikely that the model contains an indirect arc that is mediated by any molecule observed in our measurements. As can occur with closely connected pathways, correlation exists between most molecule pairs in this dataset (per Bonferroni corrected p value, see FIG. 6). Therefore, the relative “lack” of arcs in the model (FIG. 3A) contributed greatly to the accuracy and interpretability of the inferred model.

A more complex example is the influence of PKC upon Mek, known to be mediated by Raf (FIG. 3B, panel d). PKC is known to affect Mek through two paths of influence, each mediated by a different active, phosphorylated, form of the protein Raf. Although PKC phosphorylates Raf directly at S499 and S497, this event is not detected by our measurements, as we use only an antibody specific to Raf phosphorylation at S259 (Table 2). Therefore, our algorithm detects an indirect arc from PKC to Mek, mediated by the presumed unmeasured intermediate Raf phosphorylated at S497 and S499 (Jaumot, M. & Hancock, J. F. (2001) Oncogene 20, 3949-58). The PKC to Raf arc represents an indirect influence that proceeds via an unmeasured molecule, presumed to be Ras (Marshall, C. J. (1994) Curr Opin Genet Dev 4, 82-9, Carroll, M. P. & May, W. S. (1994) J Biol Chem 269,1249-56). We discuss above the ability of our approach to dismiss redundant arcs. In this case there are two paths leading from PKC to Mek because each path corresponds to a separate means of influence from PKC to Mek-one via Raf phosphorylated at S259, and the other through Raf phosphorylated at S497 and S499. Thus, neither path is redundant. This result demonstrates the important distinction that this analysis is sensitive to specific phosphorylation sites on molecules and is capable of detecting more than one route of influence between molecules.

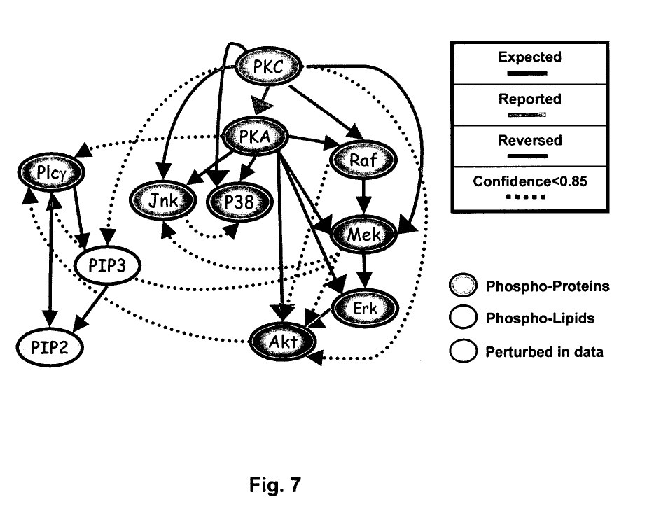

Four well-established influence connections do not appear in the model: PIP2 to PKC, PLCg to PKC, PIP3 to Akt, and Raf to Akt. Bayesian networks are constrained to be acyclic, so if the underlying network contains feedback loops we cannot necessarily expect to uncover all connections (FIG. 7). For example, in our model the path from Raf to Akt (via Mek and Erk) precludes the inclusion of an arc from Akt to Raf, due to this acyclicity constraint. Availability of suitable temporal data could possibly permit this limitation to be overcome using dynamic Bayesian networks (Fortino, V., Torricelli, C., Gardi, C., Valacchi, G., Rossi Paccani, S. & Maioli, E. (2002) Cell Mol Life Sci 59, 2165-71, and Zheng, M., Zhang, S. J., Zhu, W. Z., Ziman, B., Kobilka, B. K. & Xiao, R. P. (2000) J Biol Chem 275, 40635-40).

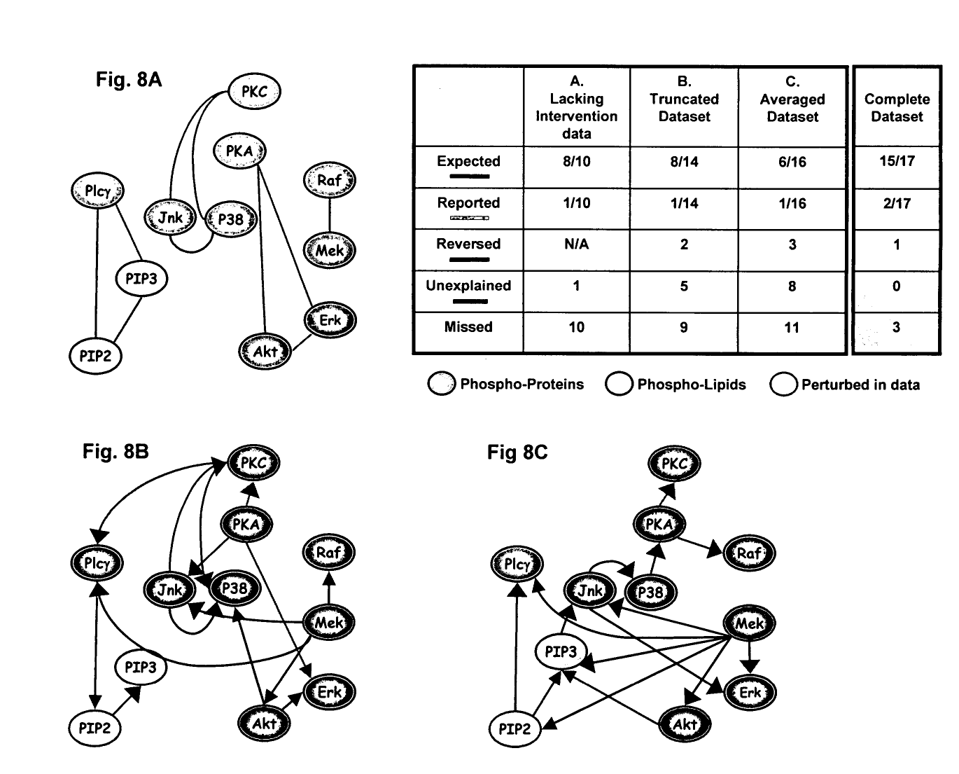

Three influence connections in the model are not well established in the literature: PKC on PKA, Erk on Akt, and PKA on Erk. To probe the validity of these proposed causal influences, we searched for prior reports in the literature. Of these 3 connections, 2 have previously been reported, the PKC to PKA connection in rat ventricular myocytes, and the Erk to Akt connection in colon cancer cell lines (Clerk, A., Pham, F. H., Fuller, S. J., Sahai, E., Aktories, K., Marais, R., Marshall, C. & Sugden, P. H. (2001) Mol Cell Biol 21, 1173-84, Zhang, W. M. & Wong, T. M. (1998) Am J Physiol 274, C82-7). An important goal is to test the ability of Bayesian network analysis of flow cytometry data to correctly infer causal influences from unperturbed molecules within a network. For example, Erk was not acted upon directly by any activator or inhibitor in the sample sets, yet Erk showed an influence connection to Akt. The model thus predicts that direct perturbation of Erk would influence Akt (FIG. 8A). On the other hand, although the Erk and PKA are correlated (see FIG. 6), the model based on Bonferoni corrected p value predicts that perturbation of Erk should not influence PKA.

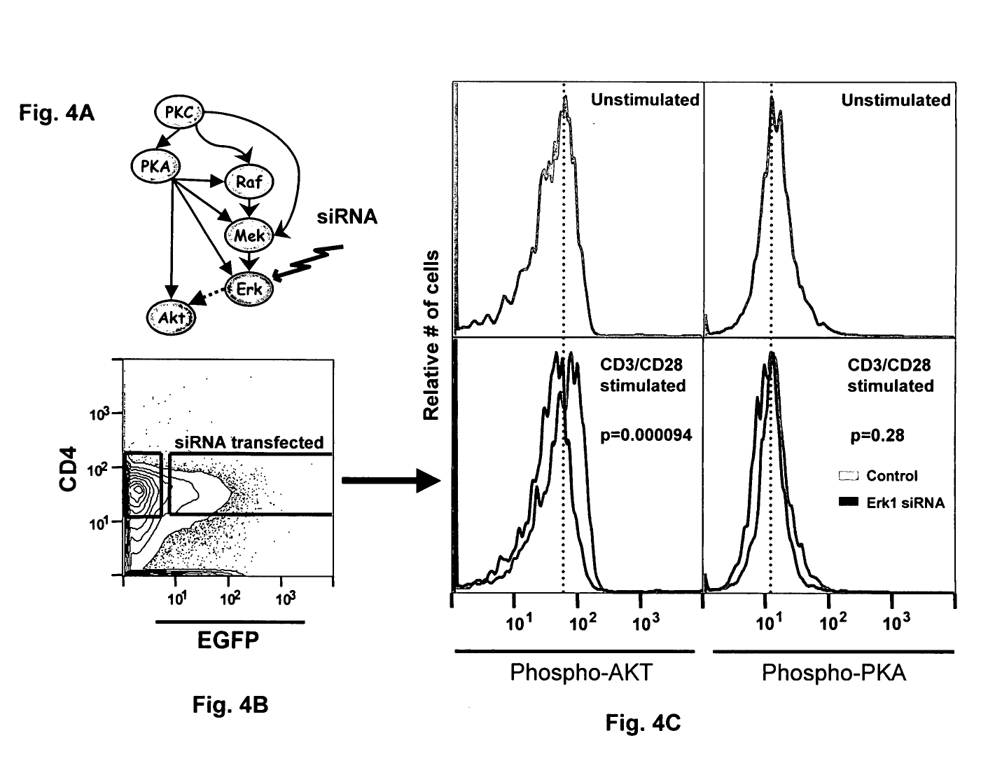

As a test of these predictions (FIG. 4A), we used siRNA inhibition of either Erk1 or Erk2 and the amount of S473 phosphorylated Akt and phosphorylated PKA were then measured. In accord with the model predictions, Akt (p<9.4e⁻⁵) phosphorylation was reduced after siRNA knockdown of Erk1 but activity of PKA (p<0.28) was not (FIGS. 4B and 4C). Akt phosphorylation was not affected by the knock down of Erk2. The connection between Erk 1 and Akt may be direct or indirect, involving mediatory molecules yet to be understood, but the connection is supported by both the model and the validation experiment.

Three features distinguish our data from the majority of currently attainable biological datasets. First, we measured multiple protein states simultaneously in individual cells, eliminating population averaging effects that could obscure interesting correlations. Second, because the measurements are on single cells, thousands of data points were collected in each experiment. This feature constitutes a tremendous asset for Bayesian network modeling, as the large number of observations allows accurate assessment of underlying probabilistic relationships, and hence extraction of complex relationships from ‘noisy’ data. Third, interventional assays generated hundreds of individual data points per intervention (because flow cytometry measures single-cells in population), allowing for an increase in inferences of causality. To evaluate the importance of these features, we created variations on our original data-set: [i] an observation-only dataset (that is, without any interventional data) of 1200 data points; [ii] a population-averaged (that is, a simulated western blot) dataset and [iii] a truncated individual-cell dataset of size comparable to the simulated western blot dataset (that is, the original dataset with most of the data randomly excluded to reduce its size, see Methods). Bayesian network inference was performed on each set of data. The network inferred from 1200 observational data points included only 10 arcs, all undirected, of which 7 were expected or reported, and 11 arcs were missing (FIGS. 4A-4C). This demonstrates that interventions are useful for effective inference, particularly to establish directionality of the connections (see also FIG. 1B). The truncated single cell dataset (420 data points) shows a large (11-arc) decline in accuracy, missing more connections and reporting more unexplained arcs than its larger (5400 data points) counterpart (FIG. 8A). This result emphasizes the importance of sufficiently large dataset size in network inference. The network inferred from averaged data (FIG. 8C) shows a further 4-arc decline in accuracy relative to that inferred from an equal number of single cell data points, emphasizing the importance of single cell data. The fact that population averaging destroys some of the signals present in the data may reflect the presence of heterogeneous cellular subsets that are masked by averaging techniques.

As shown, using the methods described herein enabled the generation of a model for classically understood signaling network that connects a number of key phosphorylated proteins in human T cell signaling—a map built by classical biochemistry and genetic analysis over the last two decades. The network was constructed with no a priori knowledge of pathway connectivity. Thus, application of Bayesian networks to single cell flow cytometry has distinct advantages, including an ability to measure events in primary cells after in vivo interventions (thus measuring context specific signaling biology in tissues), inference of directed arcs and causality therein, and the ability to detect indirect as well as direct connections. This latter point is a powerful feature when the known list of participating molecules may not be exhaustive, and can be especially important when networks are used to assess the effects of system perturbations (as in a pharmaceutical context).

Another advantage of using Bayesian networks to model cell signaling networks is that they are relatively robust to the existence of unobserved variables, for example their ability to detect indirect influences via unmeasured molecules. At the forefront of Bayesian network research is development of methods to automatically infer the existence and location of such hidden variables. Although the current report is restricted to 11 phosphorylated molecule measurements per cell, the number of simultaneous parameters measured by flow cytometry is steadily growing (Lange-Carter, C. A. & Johnson, G. L. (1994) Science 265,1458-61, Jaiswal, R. K., Moodie, S. A., Wolfman, A. & Landreth, G. E. (1994) Mol Cell Biol 14, 6944-53). As measurement systems improve, and more probes become available to detect cellular components involved in signaling networks, the ability to readily and accurately measure greater numbers of internal signaling events increases, providing additional opportunities to discover novel influences and pathway structures.

Materials and Methods

Reagents. Protein and chemical reagents used (and vendors) were as follows: 8-Bromo-cAMP (8-bromo Adenosine 3′,5′-cyclic Monophosphate, b2cAMP), AKT inhibitor, G06976, LY294002, psitectorigenin and U0126: Calbiochem. PMA: Sigma. Recombinant human ICAM2-FC was produced as reported (1). Alexa fluor dye series (488, 546, 568, 594, 633, 647, 680), cascade yellow, cascade blue, allophycocyanin (APC), and R-Phycoerythrin (PE): Molecular Probes; cyanine dyes (Cy5, Cy5.5, Cy7: Amersham Life Sciences. Tandem conjugate protocols for PECy5, PECy5.5, PECy7, APCCy5.5, and APCCy7 are readily available. a-CD3 (clone UCHT1) and a-CD28 (clone 28.2): BD-Pharmingen; antibodies to phosphoproteins Raf-259, Erk1/2-T202/T204, p38-T180/Y182, Jnk-T183/Y185, Akt-S473, Mek1/2-S217/S221, PKA substrates (a measure of PKA activation), PKC-S660, and Plcg-Y783: Cell Signaling Technologies; antibodies to PIP2 and PIP3: Molecular Probes; antibodies to Erk1/2-T202/T204-phycoerythrin and PKA-S114: BD-Pharmingen. Phospho-AKT-S473 in FIG. 3 was from Biosource.

Cell culture. Human peripheral blood lymphocytes were obtained by Ficoll-plaque density centrifugation (Amersham Pharmacia, Uppsala, Sweden) of whole blood from healthy donors (Stanford Blood Bank) and depleted for adherent cells. Magnetically activated cell sorting was used to negatively isolate naive CD4+ cells (Dynal, Oslo, Norway). Human cells were maintained in RPMI-1640 supplemented with 5% human sera AB (Irvine Scientific), and 1% PSQ (1000 units penicillin supplemented with 2 mM L-glutamine). Cells were maintained at 5% CO2 at/370 C in a humidified incubator.

Flow cytometry. Intracellular and extracellular staining was performed as described (Perez, O. D. & Nolan, G. P. (2002) Nat Biotechnol 20, 155-62). Intracellular probes for active kinases were made by conjugating phospho-specific antibodies to the Alexa Fluor dye series as described and used in phospho-protein staining (Perez, O. D., Krutzik, P. O. & Nolan, G. P. (2004) Methods Mol Biol 263, 67-94, Perez, O. D. & Nolan, G. P. (2002) Nat Biotechnol 20, 155-62). Briefly, purified human CD4+ T cells were dispensed in 96 wells, and treated with chemical inhibitors for 30 min, then were treated with stimulatory agents for 15 min. Analyses were performed by direct application of fixation buffer to time-synchronized 96-wells (i.e. a single 96-well plate) maintained at 37° C. 2% paraformaldehyde (200 uL) was added to 0.5×106 cells (in 100 uL), stimulated as indicated. Fixation was performed for 30 min on pre-chilled 96-well metal holders at 40° C. Plates were then centrifuged (1500 RPM, 5 min, 40° C.) and stained with pre-titred multi-color antibody cocktails. Cells were washed three times and analyzed. Flow cytometry data are representative of at least 3 three independent experiments. Data were collected on a custom-configured machine, a modified FACStar bench (Becton Dickenson) connected to MoFlo electronics (Cytomation, Fort Collins Colo.) (Tung, J. W., Parks, D. R., Moore, W. A. & Herzenberg, L. A. (2004) Methods Mol Biol 271, 37-58). This configuration allows for 11-color analysis of samples and real-time compensation for spectral overlap (plus two channels for forward and side scatter). Data was collected using Desk software (Stanford University), compensated (intra-laser and fluorophore spectral overlap demixing) and analyzed using Flowjo software (Treestar).

siRNA inhibitions. siRNA complementary to Erk1 mRNA was purchased from Superarray Biosciences. siRNA complementary to Erk2 mRNA was purchased from Upstate Biotechnologies. siRNA oligonucleotide (100 nM) was used in primary cell transfections using the Amaxa nucleofector systems (Amaxa Biosystems) (Lenz, P., Bacot, S. M., Frazier-Jessen, M. R. & Feldman, G. M. (2003) FEBS Lett 538,149-54).

Conditions employed. The following conditions were used for model inference: 1: (anti-CD3 and anti-CD28), 2: (anti-CD3, anti-CD28 and Intercellular Adhesion Protein-2 (ICAM-2) protein), 3: PMA (phorbol myristate acetate), 4: b2cAMP (8-bromo Adenosine 3′,5′-cyclic Monophosphate), 5: (anti-CD3, anti-CD28 and U0126), 6: (anti-CD3, anti-CD28 and G06976), 7: (anti-CD3, anti-CD28 and Psitectorigenin), 8: (anti-CD3, anti-CD28 and Akt-inhibitor), and 9: (anti-CD3, anti-CD28 and LY294002). Each condition provided 600 cells, for a total of 5400 datapoints. For the simulated western blot dataset and its single-cell equivalent, the following conditions were also used: 1 (anti-CD3, anti-CD28, ICAM2 protein and U0126), 2 (anti-CD3, anti-CD28, ICAM2 protein and G06976), 3 (anti-CD3, anti-CD28, ICAM2 protein and Akt-inhibitor), 4 (anti-CD3, anti-CD28, ICAM2 protein and Psitectorigenin,) and 5 (anti-CD3, anti-CD28, ICAM2 protein and LY294002). Equal numbers of cells (600) were selected at random from each condition, to prevent biasing the network to any particular condition.

Processing of data. Data were preprocessed as follows: Data points that fell more than three standard deviations from the mean were eliminated. Data were then discretized to three levels (low, medium or high levels of the phosphorylated protein), using an agglomerative approach that seeks to minimize loss of pairwise mutual information among variables (Hartemink, A. J. & Massachusetts Institute of Technology. Dept. of Electrical Engineering and Computer Science. (2001), pp. 206). Under conditions of chemical intervention, inhibited molecules were set to level 1 (‘low’), activated molecules were set to level 3 (‘high’).

Simulated western blots. To create a simulated western blot dataset, the following was repeated for each condition: 20 cells were selected at random and averaged, until all the cells had been averaged (yielding 30 simulated western blot datapoints per condition). Averaging reduces the size of the dataset to 1/20th of the original size, therefore 5 additional conditions containing ICAM2 (see above) were used to create the simulated western blot dataset, for a total of 420 datapoints. For a single cell dataset of equivalent size, 30 cells were selected at random from each of the 14 conditions. This process was repeated 10 times, each with a different random seed, producing 10 different simulated western blot and truncated datasets. The Bayesian network inference procedure (see below) was independently applied to each such dataset.

Bayesian network structure inference. We implemented Bayesian network inference as described in the specification and in Pe'er, D., Regev, A., Elidan, G. & Friedman, N. (2001) Bioinformatics 17 Suppl 1, S215-24, and Yoo, C. a. C. G. F. (1999) in Uncertainty in Artificial Intelligence, pp. 116-125, the disclosures of which are incorporated herein by reference. See also Friedman (Friedman, N. (2004) Science 303, 799-805), incorporated herein by reference, for a review on the methodology.

1. A method of developing a model of cellular networks within a first cell category comprising:
a) contacting first cells of said first cell category with a set of probes that bind to a set of cellular components in each of said first cells, wherein each probe is labeled with a distinguishable label;
b) detecting a plurality of said cellular components in each of said first cells to generate a first data set associated with said cellular components in each of said first cells; and
c) applying a probabilistic graphical model algorithm to said first data set to identify a first set of arcs between individual cellular components in each of said first cells.

2. A method according to claim 1 wherein said detecting step comprises a detection technique selected from the group consisting of flow cytometry and confocal microscopy.

3. The method of claim 1, wherein the probabilistic graphical model algorithm is selected from the group consisting of a Bayesian network structure inference algorithm, a factor graph, a Markov random fields model, and a conditional random fields model.

4. The method of claim 3, wherein the probabilistic graphical model algorithm is a Bayesian network structure inference algorithm.

5. A method according to claim 1 in which said known cellular components comprise one or more proteins.

6. A method according to claim 5 in which one or more of said proteins is a kinase.

7. A method according to claim 5 in which one or more of said proteins is a phosphatase.

8. A method according to claim 1 in which said cellular components comprise one or more substrate molecules.

9. A method according to claim 1 in which said known cellular components comprises one or more non-protein metabolites.

10. A method according to claim 9, wherein said non-protein metabolites are selected from the group consisting of carbohydrates, phospholipids, fatty acids, steroids, organic acids, and ions.

11. The method of claim 1, wherein one or more of said arcs is identified between one of said cellular components bound by one of said probes and a cellular component not bound by one of said probes.

12. The method of claim 1, wherein one or more of said arcs is identified between at least two of said cellular components bound by said probes.

13. A method of characterizing a disease state comprising:
a) providing a first set of arcs for a set of cellular components from measurements of individual cells exhibiting said disease state;
b) providing a second set of arcs for said set of cellular components from measurements of individual cells that do not exhibit said disease state; and
c) comparing said first and second sets of arcs to determine one or more decisional arcs indicative of said disease state.

14. A method of diagnosing a disease state in a subject comprising:
a) providing a set of decisional arcs indicative of the presence or absence said disease state;
b) obtaining a first set of cells from said subject;
c) providing a set of probes that bind to a set of cellular components in said first set of cells, wherein each probe is labeled with a distinguishable label;
d) detecting a plurality of said cellular components in each individual cell of said first set of cells to generate a first data set associated with said cellular components in each of said first cells; and
e) applying a probabilistic graphical model algorithm to said first data set to identify a set of arcs between individual cellular components in each said cell, wherein said set of arcs corresponds to said set of decisional arcs; and
f) comparing said set of arcs to said set of decisional arcs to diagnose said disease state in said subject.

15. A method of prognosing a disease state in a subject comprising:
a) providing a set of decisional arcs indicative of a prognosis of said disease state;
b) obtaining a set of cells from said subject;
c) providing a set of probes that bind to a set of cellular components in said set of cells, wherein each probe is labeled with a distinguishable label;
d) detecting a plurality of said cellular components in each individual cell of said set of cells to generate a data set associated with said cellular components in each of said cells; and
e) applying a probabilistic graphical model algorithm to said data set to identify a set of arcs between individual cellular components in each said cell, wherein said set of arcs corresponds to said set of decisional arcs; and
f) comparing said set of arcs to said set of decisional arcs to diagnose said disease state in said subject.

16. A method according to claim 1 further comprising;
a) contacting one or more second cells of said first cell category with an agent;
b) contacting said second cells with said set of probes;
c) detecting a plurality of said cellular components in each of said second cells to generate a second data set associated with said cellular components in each of said second cells;
d) applying a probabilistic graphic model algorithm to said second data set to determine one or more arcs between individual cellular components of said second cells; and
e) comparing said first set of arcs with said second set of arcs.

17. The method of claim 16, wherein said one or more decisional arcs identifies said agent as therapeutic to said subject.

18. The method of claim 16, wherein said one or more decisional arcs identifies said agent as toxic to said subject.

19. The method of characterizing the biochemical effects of an agent according to claim 16, wherein said first and second cell populations comprise cells from a subject with a disease state.

20. A method of identifying sub-populations of cells in a population of cells comprising:
a) developing a model of cellular networks in each individual cell in said population of cells according to claim 1 to obtain a set of one or more arcs; and
b) identifying two or more sub-populations of cells, wherein the presence, absence, or difference in one or more arcs in a first sub-population of said cells that are not present in a second sub-population of said cells to form said first and second sub-populations of cells.

21. A method of categorizing individual cells in a population of cells into one or more cell categories comprising;
a) developing a cellular network of each said individual cells in said population of cells according to the method of claim 1;
b) identifying one or more decisional arcs corresponding to each said cell category; and
c) categorizing each said cell in each of one or more categories.

22. A method of refining a model of cellular networks comprising:
a) categorizing individual cells in a population of cells into one or more sub-populations of cells according to the method of claim 21;
b) developing a cellular network In each individual cell in each said sub-population of cells according to claim 1 to refine said model of cellular networks; and
c) identifying one or more arcs characteristic of each said sub-population to define a refined model of cellular networks.

23. The method of claim 22, wherein each said subpopulation corresponds to a disease state.

24. A method of identifying one or more cellular components affected by an agent comprising: characterizing one or more biochemical effects of an agent on a population according to claim 16; identifying said one or more biochemical effects that correspond to said agent.

25. A method of determining the dose of an agent to administer to a subject comprising:
a) providing a set of decisional arcs indicative of characteristic of treatment of said disease state;
b) providing an agent to said subject;
c) obtaining a set of cells from said subject;
d) providing a set of probes that bind to a set of cellular components in said set of cells, wherein each probe is labeled with a distinguishable label;
e) detecting a plurality of said cellular components in each individual cell of said set of cells to generate a data set associated with said cellular components in each of said cells; and
f) applying a probabilistic graphical model algorithm to said data set to identify a set of arcs between individual cellular components in each said cell, wherein said set of arcs corresponds to said set of decisional arcs; and
g) comparing said set of arcs to said set of decisional arcs to determine the effectiveness of said dose.

26. The method of claim 25, further comprising altering said dose based on the effectiveness of said dose.
